(19) United States
(12) Patent Application Publication
(10) Pub. No.: US 2025/0352853 A1
(43) Pub. Date: Nov. 20, 2025

(54) A SYSTEM AND METHOD FOR MEASURING PERFORMANCE

(71) Applicant: OMNIBUS157 PTY LTD, Sydney (AU)

(72) Inventors: Kenneth Graham, Sydney (AU); Neil Baker, Sydney (AU); Kurt Friday, Sydney (AU)

(73) Assignee: OMNIBUS157 PTY LTD, Sydney (AU)

(21) Appl. No.: 18/871,097

(22) PCT Filed: May 31, 2023
(86) PCT No.: PCT/AU2023/050467

§ 371 Date: Dec. 2, 2024

(30) Foreign Application Priority Data
May 31, 2022 (AU) 2022901486

(51) Int. Cl. A63B 24/00

(52) U.S. Cl. CPC A63B 24/0062; A63B 2220/40; A63B 2220/56; A63B 2220/62; A63B 2230/62; A63B 2244/20

(57) ABSTRACT

A method of determining a performance metric of an athlete is provided. The method includes receiving, with a processing system, data from at least one limb of the athlete, determining an orientation of the at least one limb, and applying the orientation to the received data. The method also includes generating the performance metric of the at least one limb based on received data and orientation of the at least one limb.

### TECHNICAL FIELD

The present invention relates to a method and system for measuring performance. In one particular example, the present invention relates to measuring performance in water sport such as swimming, rowing, or the like.

### BACKGROUND OF THE INVENTION

The following references to and descriptions of prior proposals or products are not intended to be and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

Over time, certain performance metrics of an athlete have been measured and captured to determine areas for improvement and training. As an example in swimming, certain performance metrics are often viewed by coaches/trainers to see what area an athlete could improve in in order to then improve their overall performance. This is typically done by a coach watching an athlete and commenting on their stroke. Thus for example, in freestyle a coach may notice that when a swimmer takes a breath, the body of the swimmer rolls too much to one side. This can create unnecessary drag and slow the swimmer down. Thus in training, the coach may then suggest techniques for decreasing the body roll.

However, these techniques are often based on a coach watching an athlete and knowing instinctively what an athlete can improve on. They are typically not based on receiving objective measurements on how the swimmer is moving their body through water for forward propulsion.

The present invention seeks to provide a system and method for measuring one or more performance metrics which may ameliorate the foregoing shortcomings and disadvantages or which will at least provide a useful alternative.

### SUMMARY OF THE INVENTION

According to one aspect of the invention, there is provided herein a method of determining a performance metric of an athlete, the method including the steps of receiving data from at least one limb of the athlete; determining an orientation of the at least one limb and applying the orientation to the received data; and, generating the performance metric of the at least one limb based on received data and orientation of the at least one limb.

According to one example, the method can be performed by one or more processing systems which can be a part of a discrete or distributed/networked system.

In a further example, the method includes receiving data including any one or a combination of pressure data from the at least one limb; acceleration data of the at least one limb; and time data.

In one form, the at least one limb is a hand of the athlete and the pressure data includes receiving palm pressure of the hand and side pressure of the hand.

According to another example, the method includes determining a pressure difference, the pressure difference being difference in pressure between the palm pressure and the side pressure.

In yet another example, receiving pressure data includes receiving data from a left hand and a right hand of the athlete.

In one example, the method of determining a performance metric of an athlete, the method including the steps of, in a processing system: receiving data from at least one limb of the athlete; determining an orientation of the at least one limb and applying the orientation to the received data; and, generating the performance metric of the at least one limb based on received data and orientation of the at least one limb.

In another example, determining the orientation of the athlete includes applying a quaternion rotation to at least some of the received data. That is, applying the rotation function can include applying a quaternion rotation.

According to another form, the method includes determining any one or a combination of pressure in three-dimensions; and, acceleration in three-dimensions.

According to another example, determining pressure in three dimensions includes determining forward pressure of the limb; lateral pressure of the limb; and, vertical pressure of the limb.

In a further example, determining acceleration in three dimensions includes determining: forward acceleration of the limb; lateral acceleration of the limb; and, vertical acceleration of the limb.

In another example, the method includes determining velocity of the limb in one or more dimensions, including forward velocity, lateral velocity and vertical velocity.

In one example, the method of determining velocity includes: determining acceleration in one or more dimensions; integrating the acceleration in one or more dimensions to determine velocity in one or more dimensions.

In a further example, the method includes determining displacement of the limb in one or more dimensions, including any one or a combination of forward displacement, lateral displacement, and vertical displacement.

In one example, determining displacement in one or more dimensions includes integrating the velocity in one or more dimensions.

In one example, the athlete is a swimmer.

In yet another example, the method includes detecting a stroke event by identifying an entry point of a hand and an exit point of the hand.

According to a further example identifying the entry point and the exit point includes determining a pressure difference between pressure measured on a side of the hand and pressure measured by a palm of the hand, and a time period.

According to another example, the method includes detecting a lap event by identifying a change in forward direction.

In another example, identifying a change in forward direction includes determining forward pressure and a time period.

In another example, the method includes detecting a pull event.

According to another example, detecting pull includes identifying one or more positions within a stroke where a forward velocity is at our near zero, indicating a transition from catch to pull.

In a further example, the method includes aggregating the stroke event, the lap event, and the pull event.

In yet another example, the method includes generating a graphical representation of the stroke event, lap event and/or pull event for one or more time periods for the swimmer.

In another form, the method includes determining stroke type or swim style.

According to a further example, stroke type or swim style can include any one or a combination of freestyle, backstroke, breaststroke, butterfly, and, drills.

In yet another example, the method includes generating a graphical representation of one or more performance metrics.

According to a further example, the method includes generating one or more graphical representations including any one or a combination of: Stroke rate and force over time; Force over time showing force applied by one or more limbs of the athlete at a particular time period; Stroke path of the one or more limb over a time period; Velocity of the one or more limb over a time period; Stroke path of two or more limbs for comparison over a time period; Segmentation of stroke phases at a time period; and, Angle of attack;

In one form, the stroke rate includes strokes per minute over time.

According to another example, the graphical representation of force over time includes any one or a combination of force per stroke; force field for a limb; and, force versus time.

According to a further form, the stroke path includes depth and outsweep of the one or more limbs.

In yet another example, the comparison includes determining consistency between limbs in relation to any one or a combination of movement through the water; depth; and, outsweep.

According to a further example, segmentation of stroke phases includes generating a graphical representation showing the percentage of glide, pull, and recovery phases of a stroke.

In another form, the angle of attack includes determining the angle of a limb at a particular point in time, and the pressure that was being exerted at that time.

According to another aspect, there is provided herein a system for determining a performance metric of an athlete, the system including a sensing device, and a processing system being configured to: receive data from the sensing device being attached to at least one limb of the athlete; determine an orientation of the at least one limb and applying the orientation to the received data; and, generate the performance metric of the at least one limb based on received data and orientation of the at least one limb.

In yet another aspect, there is provided herein a processing system for determining a performance metric of an athlete, the processing system being configured to: receive data from the sensing device being attached to at least one limb of the athlete; determine an orientation of the at least one limb and applying the orientation to the received data; and, generate the performance metric of the at least one limb based on received data and orientation of the at least one limb.

According to another aspect, there is provided herein a system of determining a performance metric of a swimmer, the system being configured to receive data from at least one limb of the swimmer; determine an orientation of the at least one limb and apply the orientation to the received data; and, generate the performance metric of the at least one limb based on received data and orientation of the at least one limb.

It will be appreciated that the aspects, forms, and examples described herein can be formed in any combination.

### BRIEF DESCRIPTION OF THE DRAWINGS

The invention may be better understood from the following non-limiting description of a preferred embodiment, in which:

FIG. 1A is a flow diagram of an example aspect of the present invention;

FIG. 1B is another flow diagram of an example aspect of the present invention;

FIGS. 2A and 2B are flow diagrams for another example aspect of the present invention;

FIGS. 3A to 3F are flow diagrams showing another example process of the present invention;

FIG. 4 is a flow diagram showing another example process of the present invention; and,

FIGS. 5 to 36 are illustrations of example graphical user interfaces showing various performance metrics as generated by the system and method as described herein.

### DETAILED DESCRIPTION OF THE DRAWINGS

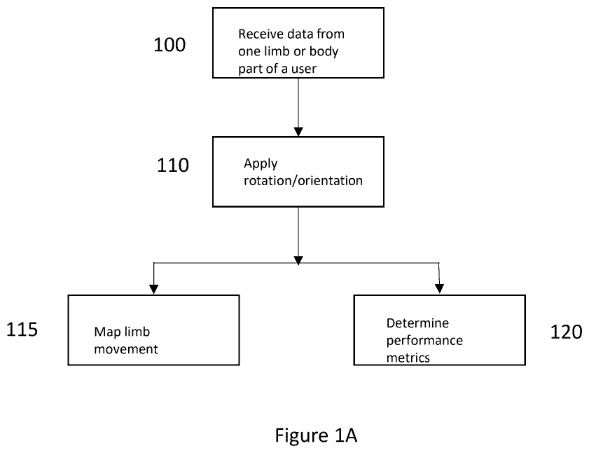

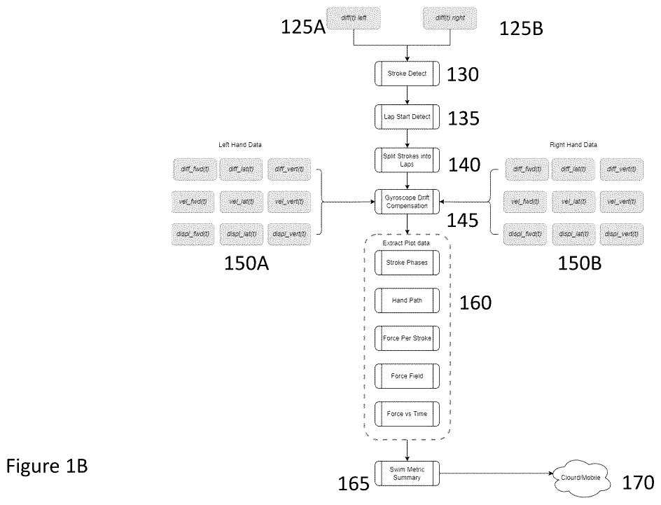

FIG. 1A shows an example of a process for generating user performance metrics. In one particular example, there is provided herein a system and method for generating a performance metric of an user or an athlete. It will be appreciated that although the examples below are provided for swimming, and in particular freestyle swimming, the system/method described herein can be applied to any form of exercise.

In the example of FIG. 1A, at step 100, data is received from at least one limb or body part of the user. It will be appreciated that although the examples herein describe data received from a hand of a swimmer, the data can be received and analysed in accordance with the system/method described herein from any suitable body part such as a leg or head of any user in a sporting activity.

As an example, the data received can include data from one or more sensing devices including one or more pressure sensors and one or more Inertial Measurement Units (IMUs), which form a part of a device which is typically attached to the at least one limb of the user (typically referred to as a hand-set device), which is configured to sense/generate various signals from one or more limbs of the user, such as pressure at certain points of the limb, acceleration, force, displacement, and the like. An example of a device is described in WO2019/204876 (“Systems and methods for formulating a performance metric of a motion of a swimmer”), the entire contents of which is incorporated herein by reference.

It will be appreciated that the performance metric can include identification of the type of stroke/swim and can thus include analytics across all strokes/drills such as, for example, stroke rate, force per stroke, distance per stroke, strokes per lap, swim time, lap time, average velocity, peak velocity, and efficiency (% forward propulsion), and as further described herein, path/trajectory the limb is moving or any form of movement data.

Once the data is received, at step 110, a rotation orientation calibration is applied to the data to determine the direction of the limb. According to one particular example, a rotation/orientation algorithm applied is applied. In one specific example, the algorithm is a quaternion rotation, although it will be appreciated by the user that any form of rotation can be applied to determine the orientation/location of the user, such as for example, a three-dimensional matrix or the like. From this, at step 115 the limb movement is visually mapped and/or various performance metrics are determined accordingly at step 120.

It will be appreciated that the process of FIG. 1A can be applied in one or more systems including one or more processing systems, which can include a networked or distributed system. As an example, a sensing device including the one or more pressure sensors/IMUs can sense data as required and communicate the data to a processing system for further processing. The processing system can be any one or a combination of hand-held device such as a tablet or smart phone, a desk-top computer or laptop, or a cloud-based system for storing and/or analysing the data and sharing the data and/or the performance metrics with other processing systems. Further, the data generated can be stored in any data store, including a database, a cloud or any distributed system.

Accordingly, there is provided herein a method for determining a performance metric of an athlete where the method includes the steps of receiving data from at least one limb of the athlete, determining an orientation of the at least one limb and applying the orientation to the received data, and generating the performance metric of the at least one limb based on received data and orientation of the at least one limb.

As described herein, the orientation of the at least one limb or the athlete can be determined initially through calibrating the sensing device which senses certain baseline performance metrics such as pressure on the at least one limb and acceleration of the at least one limb (to which the sensing device is connected).

As an example, if the athlete is a swimmer, and the sensing device is attached to one or more hands of the swimmer, the swimmer will typically stand facing the pool they are about to swim in with their palms facing up and towards the pool. Location information/data can then be received in relation to their hand with respect to the pool and thus the data from the sensing device can be altered/interpreted with respect to the pool location and orientation. In one example, a rotation algorithm such as a quaternion algorithm is applied to calibrate the location of the hand with respect to the pool. This then orientates the athlete in accordance with their location.

Thus, the system/method can receive data including any one or a combination of pressure data from the at least one limb; acceleration data of the at least one limb; and time data.

According to one example, the at least one limb is a hand of the athlete and the pressure data can include receiving palm pressure of the hand and side pressure of the hand. The method/system can then determine the pressure difference between the two sensed pressures of the palm and side of the hand. Notably, pressure data can be received from one or more limbs-thus for example, pressure data can include data from a left and a right hand of the athlete.

The method/system can then determine the pressure and/or acceleration in one or more dimensions. Typically, they are determined in three dimensions-forward, lateral and vertical (or x, y, z axes) of the limb. The rotation algorithm can thus be applied to the three dimensions to calibrate the pressure and acceleration data with respect to the location frame of reference (for example, the frame of reference of the pool).

The method/system described herein can then determine the velocity of the limb in one or more dimensions, including any one or a combination of forward velocity, lateral velocity and vertical velocity. Typically, in order to determine velocity in one or more directions, the respectively determined acceleration in one or more dimensions is integrated.

Further, the system/method can include determining displacement of the limb in one or more dimensions, including any one or a combination of forward displacement, lateral displacement, and vertical displacement. Typically, determining displacement in one or more dimensions includes integrating the respective velocity in one or more dimensions.

Notably, the method/system can also include applying trimming functions, resampling, and/or noise filters as required. Further examples of these are provided below.

Accordingly, the system/method described herein can provide certain baseline performance metrics which can be used to provide further performance analysis and graphical representation of the metric. The baseline performance metrics include and are not limited to time and pressure difference at certain points or position across a limb or body part, as well as pressure, acceleration, velocity and displacement in one or more dimensions.

In the example for swimming, the method/system described herein can also identify the entry point and the exit point of the swimmer's hand by determining a pressure difference between pressure measured on a side of the hand and pressure measured by a palm of the hand, and a time period. Further, the method/system can detect a lap event by identifying a change in forward direction, which typically includes determining forward pressure and a time period to then.

Further, the system/method can detect a pull event, which typically includes identifying one or more positions within a stroke where a forward velocity is at our near zero, indicating a transition from catch to pull. The method/system can then aggregate the stroke event, the lap event, and the pull event for a time period and can further generate a graphical representation of the stroke event, lap event and/or pull event for one or more time periods for the swimmer.

Notably, for swimming, the method/system can include determining stroke type or swim style, where the stroke type or swim style can include any one or a combination of freestyle, backstroke, breaststroke, butterfly, and, drills.

As further described herein, the method/system can include generating one or more graphical representations of the one or more performance metrics. The graphical representations can include any one or a combination of stroke rate and force over time, force over time showing force applied by one or more limbs of the athlete at a particular time period, stroke path of the one or more limb over a time period, velocity of the one or more limb over a time period; stroke path of two or more limbs for comparison over a time period, segmentation of stroke phases at a time period, and, angle of attack.

In these examples, the stroke rate includes strokes per minute over time, where the graphical representation of force over time can include any one or a combination of force per stroke, the force field for a limb, and force versus time.

Additionally, the stroke path can include depth and outsweep of the one or more limbs and comparison between limbs can include determining consistency between limbs in relation to any one or a combination of movement of the limb through the water, depth, and outsweep of the limb from the swimmer's body.

Segmentation of stroke phases can include generating a graphical representation showing the percentage of glide, pull, and recovery phases of a stroke. Furthermore, the angle of attack can include determining the angle of a limb at a particular point in time, and the pressure that is being exerted at that time.

Further examples of graphical representations are provided below.

FIG. 1B shows a further example of the process/system described herein where various performance metrics are calculated/displayed for a swim. In this example, at step 125A and 125B the time difference between the left and right hands respectively are determined in order to detect a stroke at step 130. At 135 the start of the lap is detected and at step 140 strokes are split into laps. At step 145 any filtering or calibrating of the signals received such as compensating for gyroscope drift is applied to the data received at step 150A and 150B from the left and right hands respectively. Data which is typically received from each hand for a swim can include, but is not limited to, time difference in forward, lateral, and vertical directions; velocity in forward, lateral, and vertical directions; and, displacement forward, lateral, and vertical directions.

At step 160 various data measurements are extracted and can be plotted in accordance with the data determined/received from each hand. This includes stroke phases, hand path, force per stroke, force field, and force vs time. As an example, a graphical representation of a swimmer's hand path can be generated which can show movement of the hand as it moves through a stroke. That is, how deep the hand travels into the water, and also including the velocity and displacement of the hand as the hand moves through the water. Thus, data representing each stroke movement can be mapped graphically for a user to view. Further examples of mapping of strokes is discussed below.

The user can also be provided with analysis or a swim metric summary at step 165 and the data can be stored at 170 for the user in any digital storage means/device such as either locally on a processing system such as a mobile or desktop device, or on a cloud system.

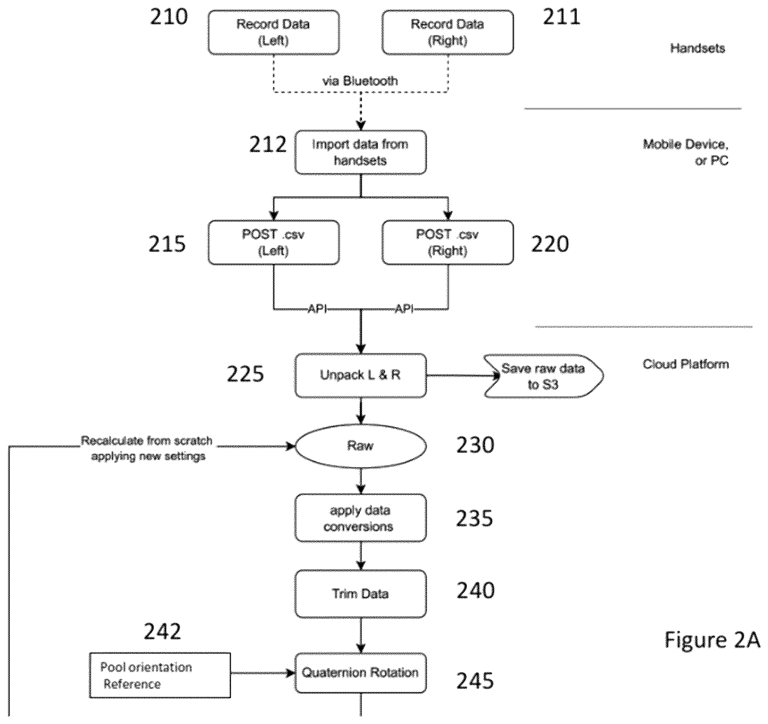

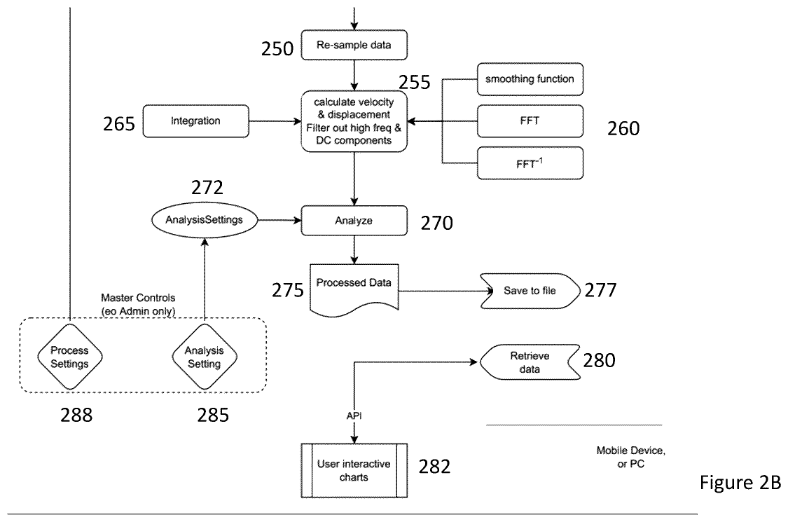

FIGS. 2A to 2B show an example of a process for generating user performance metrics of a swimmer where the one or more limbs are typically one or more hands of the swimmer.

In this example, the process includes, at step 210, data being generated/recorded from a left hand of a user and at 211 data being generated/recorded from a right hand of a user, typically by the device as described above (referred to as a handset). At step 212 the data is sent via a communication system (such as Bluetooth or any wireless communication system or the like) to a processing system such as a mobile telecommunication device or a personal computer, or the like. At steps 215 and 220 the data received is extracted separately from the left and the right by the processing system and then the data is unpacked at step 225 and presented as raw data at step 230. At step 235 the data is converted. For example, pressure data received from the handset is typically converted to kilopascals and time is typically converted to milliseconds.

At step 240, the data received is often trimmed. This can include, for example, determining the first water entry and removing all data leading up to the start of the swim, which is typically not required for determining a performance metric.

At step 245 a quaternion rotation is applied to the received data to determine the direction of the hand. This is typically through a quaternion multiplication of the data, with respect to a pool orientation reference shown at step 242.

At step 250 the data can be re-sampled, which can allow for generating data at even time intervals by taking any unevenly spaced raw data samples and re-sample the data at even time intervals. The process then continues to step 255 which includes calculating velocity and/or displacement of the hand, which includes an integration step at step 265 and at step 260 a filter is applied to the data such as a spectral filter, smoothing function or a fast fourier transform in order to smooth the data and take out any noise.

At 270 the data is analysed to determine one or more performance metrics and the process data can then be saved at step 275. Thus, at step 280, data can be retrieved to provide interactive charts/maps of the limb in motion at step 282.

Notably at step 288 and 285 process settings and analytics settings can be set respectively depending on one or more users of the system.

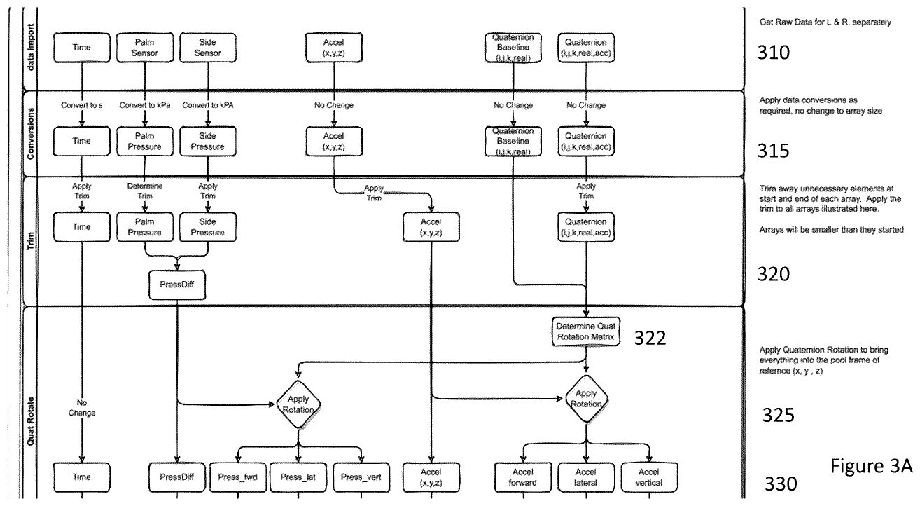

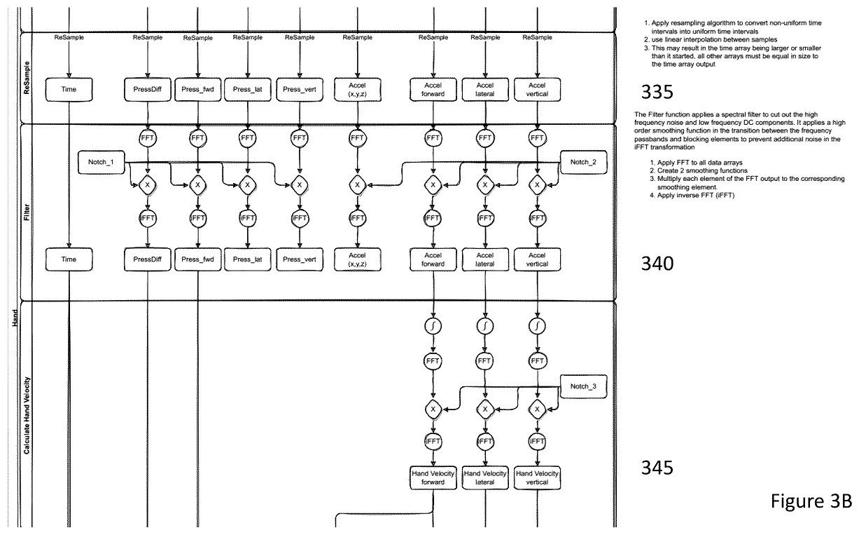

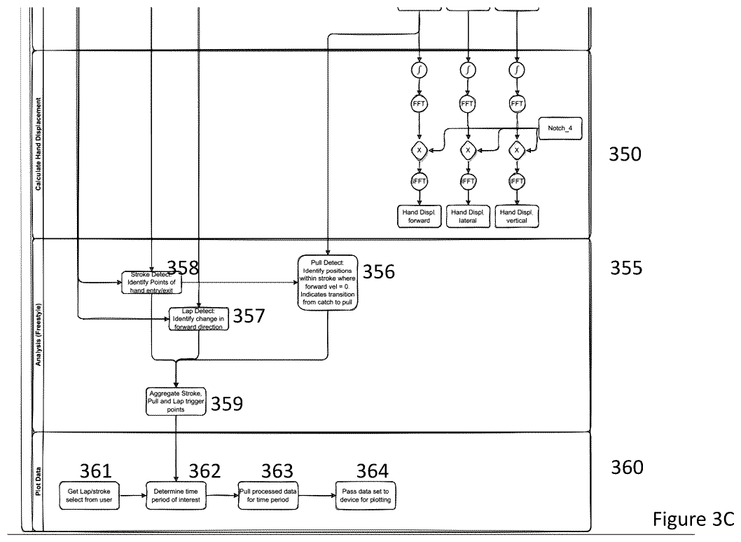

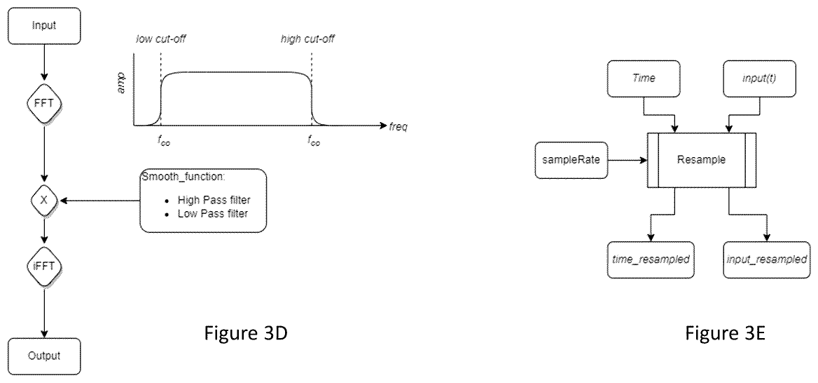

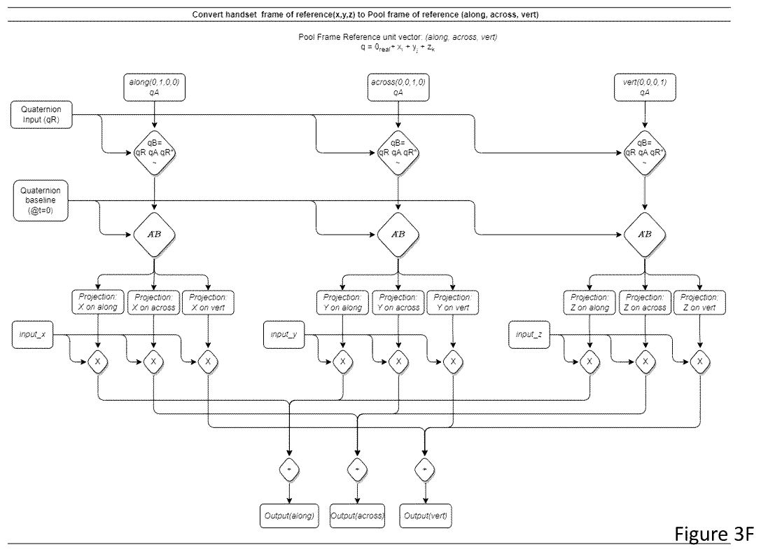

FIGS. 3A to 3C show a more detailed example of the data that can be retrieved from one or more handsets, typically attached to one or more hands of a swimmer, in order to determine one or more performance metrics.

At step 310 raw data can be received by the system for both the left and right hand. The raw data typically includes time data, at least two pressure sensors from different location of the limb (for example, a palm sensor and a side sensor of the hand), accelerometer data in one or more directions (typically in three dimensions-x, y, z), and can further include gyroscopic data in one or more directions, and a magnetometer data in one or more directions, as well as a quaternion baseline. Notably, in these examples, the directions are generally in x, y, z directions based on the direction of the swim (that is, which way the swimmer is facing in the pool).

At step 315, the data is converted as described above to a metric as required by the process, such as converting time to seconds and the pressure received to kilopascals. At step 320 the data is trimmed to only necessary data elements. Notably at this step the pressure difference between the palm and side pressures of the hand are also determined. That is, depth induced pressure can be balanced out with movement pressure to infer a force data.

At step 325 a quaternion rotation is applied to the raw data. That is, typically raw data is in the reference of the handset device, and rotation allows the data to be re-calibrated with respect to the pool's (x, y, z) frame of reference (forward, lateral, vertical). It will be appreciated that the rotation can allow for performance metrics to be determined in the direction of the swimmer's swim.

Thus, for example, at this stage the direction that the hand/palm is facing in respect of the pool can be determined, which for example, can allow for a magnitude of force in different directions can also be determined. Application of the quaternion rotation can generate, at step 330, pressure in forward, lateral, and vertical directions, as well as acceleration in forward, lateral and vertical directions.

At step 335 a re-sampling algorithm can be applied such as a linear regression between uneven data samples to interpolate the value at a desired point in time to thus generate data at uniform time intervals. Thus, for example, the resampling algorithm can convert non-uniform time intervals into uniform time intervals, can include using linear interpolation between samples. This can result in all arrays being equal in size to the time array output which can thus assist in the analysis of the data generated.

At step 340 a filtering algorithm is applied to remove any unnecessary noise from the data, such as high frequency noise and/or low frequency DC components. This can include applying a fast Fourier transform and/or a smoothing function. Typically, the smoothing function is a Low-Pass notch filter with 3 functions which can:
as a low pass filter zero out any high frequency ‘noise’ in the sampled data;
as a notch filter, can zero out the lowest frequency (DC) components; and,
as a smoothing function, can ensure the zeroing edges of the filter are smooth which helps reduce adding noise back into the data following the inverse FFT.

Furthermore, an inverse Fourier transform can also be applied to convert the time based data into frequency domain data such that the high frequency “noise” and the low frequency DC components can be separated and thus any unwanted, parasitic components of the smoothing functions can be nullified. The inverse FFT takes the filtered data and re-converts the data back into the time domain but where the noise has been removed.

Thus, for example, step 340 shows that each of the data generated including the pressure difference, forward pressure, lateral pressure, vertical pressure, acceleration in three dimensions (x, y, z), forward acceleration, lateral acceleration, and vertical acceleration can have applied thereto a filter function including applying a Fast Fourier transform (FFT) to all of the data arrays, creating two smoothing functions (referred to in FIG. 3B as Notch_1 and Notch_2, multiplying each element of the FFT output with a corresponding smoothing element, and applying an inverse FFT (iFFT) to generate filtered data of each of the pressure difference, forward pressure, lateral pressure, vertical pressure, acceleration in three dimensions (x, y, z), forward acceleration, lateral acceleration, and vertical acceleration. At step 345 performance metrics of a hand such as velocity (in three dimensions) including forward velocity, lateral velocity and vertical velocity can then be determined accordingly from the data that has been received by the handsets. In this particular example, each of the forward hand velocity, lateral hand velocity, and vertical hand velocity are determined by taking the respective forward acceleration, lateral acceleration, and vertical acceleration and applying an integration function to each acceleration direction, and further applying a filtering function by applying a FFT, multiplying by a smoothing function (Notch_3) and applying an inverse FFT.

Step 350 shows an example of determining displacement of the hand in three dimensions, that is. In this example, each of the determined forward acceleration, lateral acceleration, and vertical acceleration is taken, integrated, and a filtering function is applied thereto, which includes applying a FFT, multiplying with a smoothing function (Notch_4), and applying an iFFT to then determine the respective displacement in forward, lateral and vertical directions.

Step 355 shows an example of further performance metrics that can be derived depending on the swimming style/stroke. In these examples, the swimmer is swimming freestyle which has particular characteristics that can be determined to derive performance metrics. At 356, a pull portion of the stroke is detected by identifying, for example, positions within the stroke where the forward velocity is at or near zero, which typically indicates a transition from a catch portion of the stroke to the pull.

A stroke itself can be determined at step 358 where it is determined at what point the hand of the swimmer exited/entered the water. This is typically derived from pressure difference and time. For example, pressure on both sensors on a hand read zero when the device is out of the water as typically pressure increases with depth and/or an increased force on the palm of the hand. Thus, for example, at the start of a swim an assumption is made that the swimmer is above water when they take their ‘swim direction’ reference. Further, reference atmospheric pressure is also measured at this point. When the hand enters the water the pressure on either sensor will increase considerably due to surrounding hydrostatic pressure. Hand entry can be determined when the pressure on either sensor increased by a set threshold above the nominal atmospheric pressure. Further, hand exit can be determined when the pressure approaches atmospheric pressure.

At 357, it can be determined the swimmer has swam a lap (or is in the transition or roll between laps) by determining a change in the forward direction of the swimmer which typically includes looking at forward pressure and time. As an example, when a swimmer starts swimming forward, the pressure they exert is directed towards their feet. When they get to the end of the lap, turn and begin swimming in the opposite direction, the primary force detected is being applied in the opposite direction. This can further be determined by measuring the average period of time that a swimmer is swimming in a direction.

Then at step 359, the stroke, pull and lap trigger points can be aggregated and displayed at 360 for a particular point in time or time period. In one example, the system/method can receive a selection of the lap/stroke from a user at step 361, determine the time period of interest at step 362, pull processed data from the time period at step 363, and either pass the data to a device for plotting at step 364 or show the user a plot that has been determined/generated. Further examples of display are described below.

FIG. 3D is a further example of the noise/notch filter as described in FIGS. 3A-3B. As shown in FIG. 3D, the noise filter calculates a FFT of the data set, generates a spectral filter and overlays the spectral filter to the FFT data to suppress undesired frequency bands. The process then applies the inverse FFT to restore the data to the time base.

FIG. 3E shows a further example of the resampling as described in FIGS. 2B and 3B. In this particular example, resampling takes a non-uniformly spaced input time array and generates uniformly spaced data points at a specified sampling rate. Thus, when new time points fall between measured samples, a linear fit between the two adjacent data points can be taken to interpolate the new data point. Typically, the size of the time_resampled output array may be different to the input array, however, all resampled output arrays are typically the same size to allow further data processing.

Further detail of the quaternion rotation/transformation used is shown in FIG. 3F. In one example, quaternion rotation is used to convert the handset frame of reference (x, y, z) to a pool frame of reference (along, across, vertical). Typically the calibration for a swimmer starts prior to the swim, where the swimmer holds their handset device out in the direction of the swim.

The quaternion function can transform acceleration inputs in the handset frame of reference to the frame of reference of the pool when the user is swimming. As an example, a unit vector in each of the x, y, z directions of an IMU chip or the like can be projected onto the along, across, vertical axis of the pool frame of reference, based on the quaternion output of the IMU.

Notably, a vector in three-dimensional space can be expressed as a pure quaternion, a quaternion with no real part: q=0+xi+yj+zk. A rotation is typically expressed by a quaternion qR with the additional requirement that its normal jqRj be equal to 1. A rotation from one coordinate frame A to another B is given by the conjugation operation: qB=qE qAqR*. The quaternion qB is also a vector.

Thus, for example, before a user starts their swim, they would typically hold their palms facing up and towards the far end of the pool in order to calibrate the IMU. This starting position typically represents the quaternion baseline for the user. Thus, when the user moves their hands in their swim, all positional data determined by the sensors on the user's hands will be aligned in relation to the swimming pool by applying the quaternion rotation.

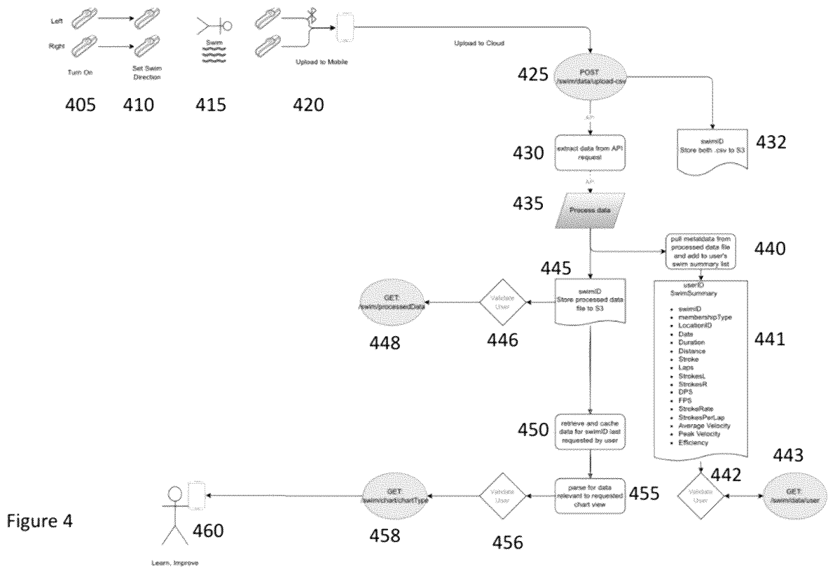

FIG. 4 shows an example of how one or more users can interact with the system and method described herein.

In this example, the user is a swimmer and has access to the system/method described herein via a software application on their mobile telecommunication device. At step 405, the user wears a handset on both their right and left hand and orientates the device so that the direction of swim is determined at step 410. Typically, orientating the swimmer initially provides a baseline measurement in which the system assesses forward/backward, up/down, left/right and generates a ‘zeroing’ vector, which can be used to track the direction a swimmer's palms are pointing.

Once the user has swum at 415 (or during the swim), the device uploads the swim data to the user's mobile telecommunication device at step 420, which can communicate with a central processing system for processing and a central data store for storage (which in this example is a cloud application/server system). At step 425 the data is posted against the user's account, using a unique SwimID at step 432.

At step 430, the system can extract the data that has been posted against the user's SwimID and process the data at step 435 by any of the methods described herein to provide the swimmer with performance metrics. As an example, at step 440, metadata/data in respect of a user and their swim can be pulled from the processed data and shown/stored in a swimmer's account on the system (typically against a User ID). The metadata can include any one or a combination of SwimID, membership type, locationID, date of swim, duration, distance, strokes (both left and right), laps, Distance per Stroke (DPS), Force per Stroke (FPS), stroke rate, strokes per lap, average velocity, peak velocity, and efficiency. Notably the metadata is thus validated against the user at 442 and stored against a user profile/ID at step 443. Similarly with the SwimID at step 445, 446 and 448.

If a user requests data for a swim, this can be accessed requesting the data for a particular SwimID at step 450. Typically the data is stored/cached against the user's SwimID. At step 455, the user may request a particular chart view or graphical representation of a performance metric. This can be requested, validated for a user at 456, generated at 458 and received by the user for viewing at 460.

### Further Examples

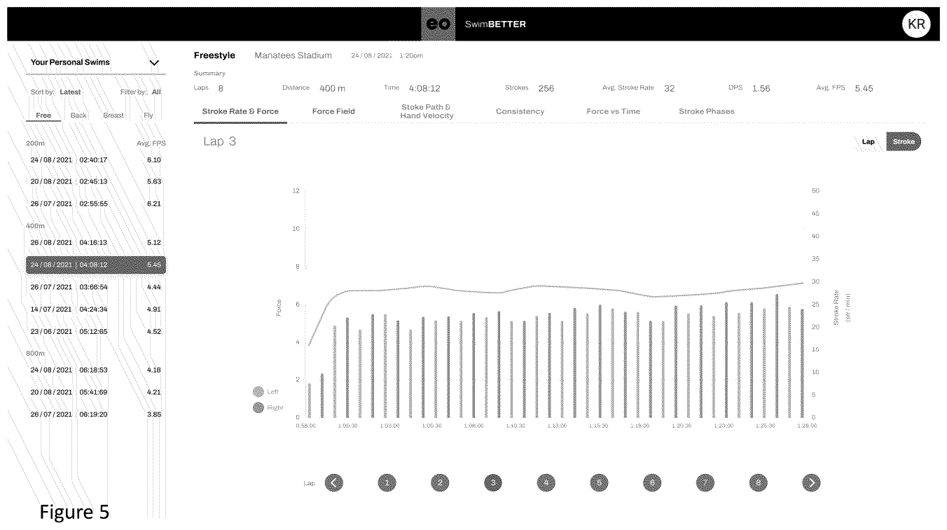

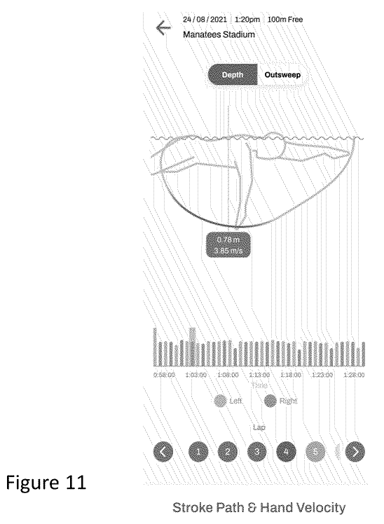

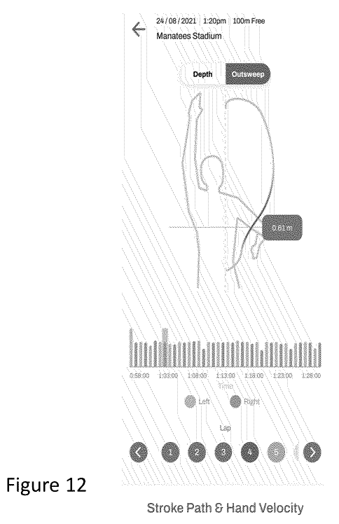

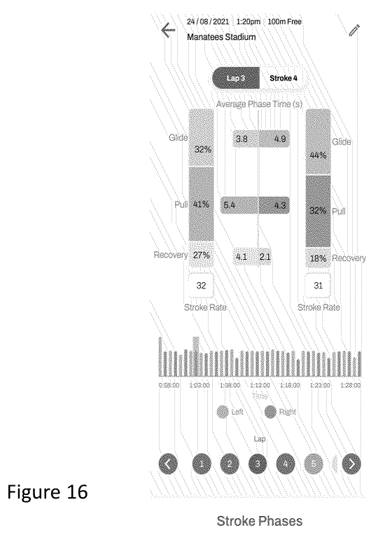

FIGS. 5 to 22 show example display images of some of the performance metrics generated in graphical or visual formats. Displaying of the performance metrics can allow for a user such as an instructor or a coach (as well as the athlete themselves) to visualise either through images of their stroke or via graphical imagery the various performance metrics for one or more swims in order to allow the user to improve their technique. It will be appreciated that the displays generated can also allow a coach or a team to view/compare metrics to improve techniques accordingly.

It will also be appreciated by a person skilled in the art that the display can be on any processing system such as for example, a desktop computing system, a mobile telecommunication device, a tablet device, or the like.

Further, FIGS. 5 to 22 show examples of freestyle swimming. However, it will be appreciated by persons skilled in the art that the same/similar methods and systems can be applied to other swimming strokes and further to other water supports such as rowing or kayaking where the handset is attached to an oar or blade.

In particular referring to FIGS. 5 to 10, a user of the system can be shown a dashboard of swimming metrics displayed graphically and can toggle between the metrics via a navigation bar or the like.

FIG. 5 shows an example of stroke rate and force of both left and right hands. The stroke rate in this example has been mapped against the force being applied by the swimmer. Also selected in this Figure is the lap that the swimmer is on. Thus, a user of the system/method described herein can readily compare a swimmer's performance across various laps.

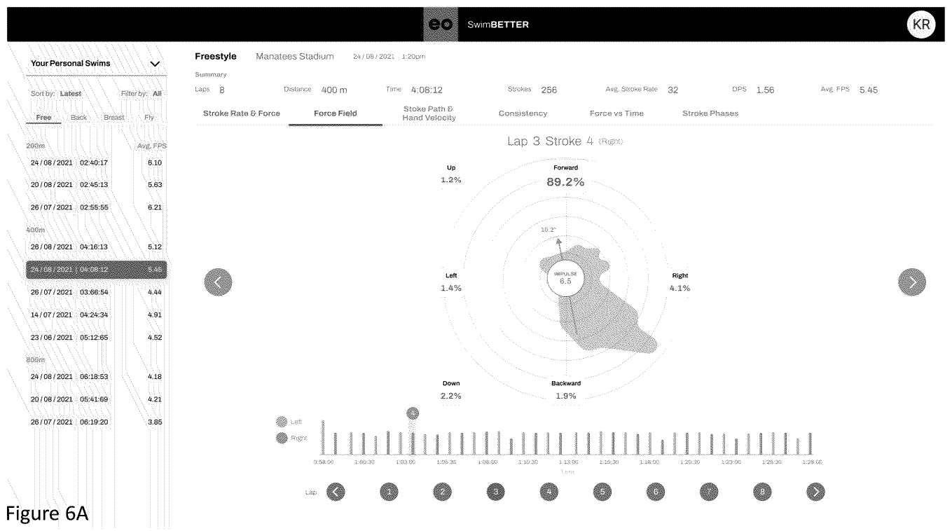

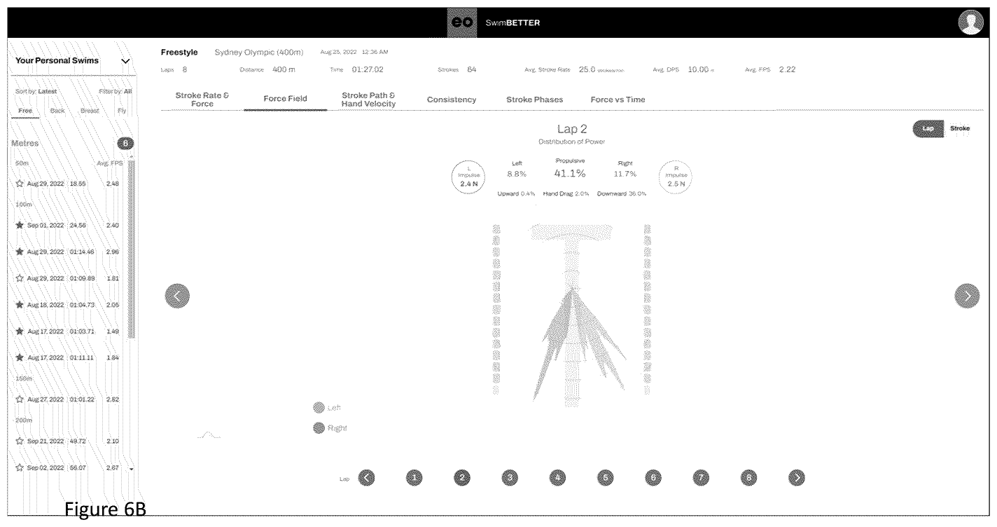

FIG. 6A shows an example force field mapped for a stroke of the right hand from above. The force field is shown for a selected time period and is split in accordance with a percentage or portion of force applied in four different directions (that is, in forward, backward, left and right directions). FIG. 6B is an example of the distribution of power between left and right hands being mapped as a part of the force field.

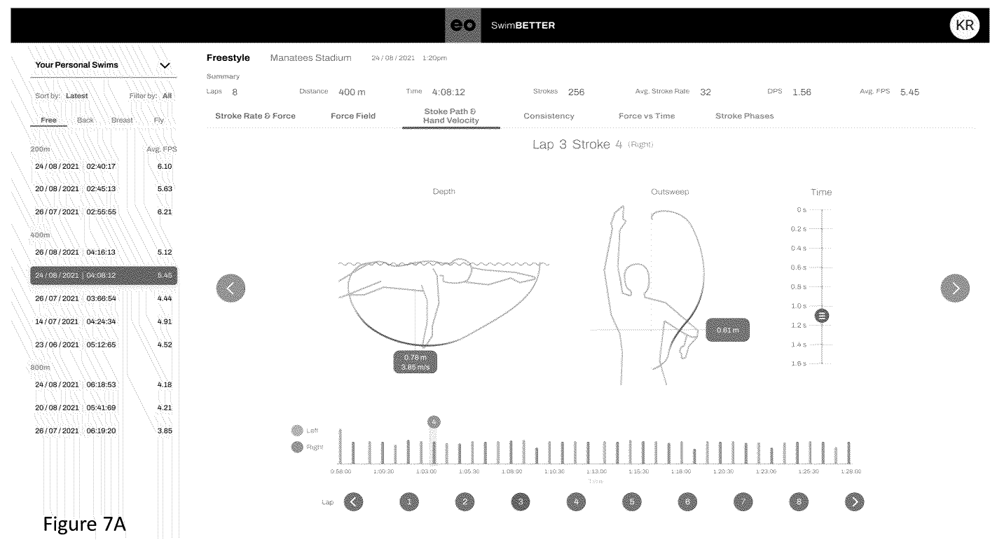

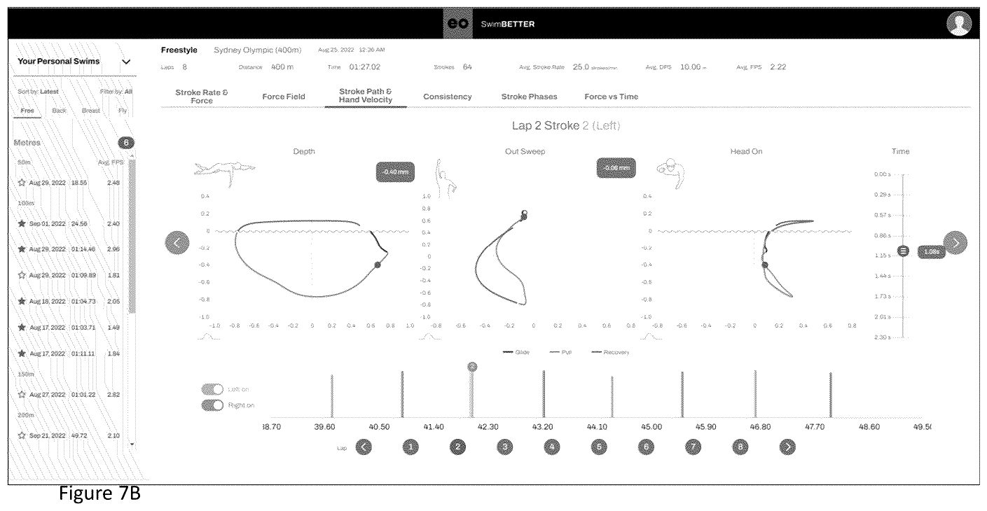

FIGS. 7A, 11, and 12 show an example of stroke path and hand velocity. The stroke path in particular can be indicative of the depth of the hand as well as outsweep. This example is for one hand only—the right hand, shown at a particular time interval and lap.

FIG. 7B is another example of stroke path and hand velocity showing three different views-depth, outsweep and a head-on view. The stroke in this map is segmented into glide, pull and recovery so that it can be determined how much of the stroke is in each phase.

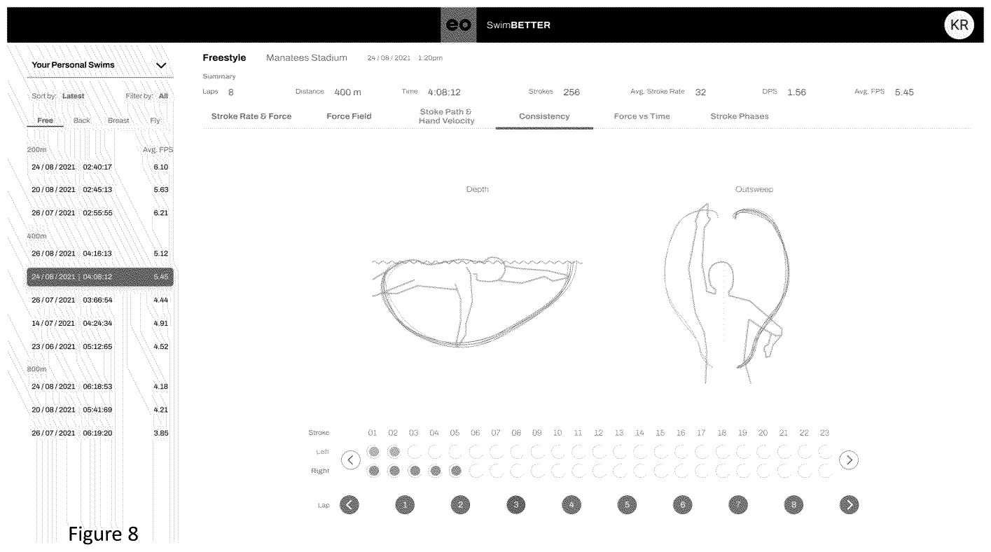

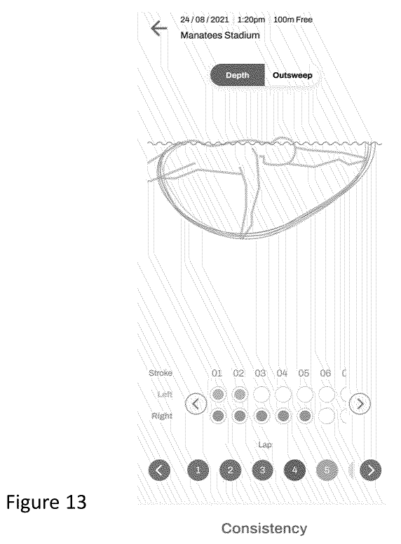

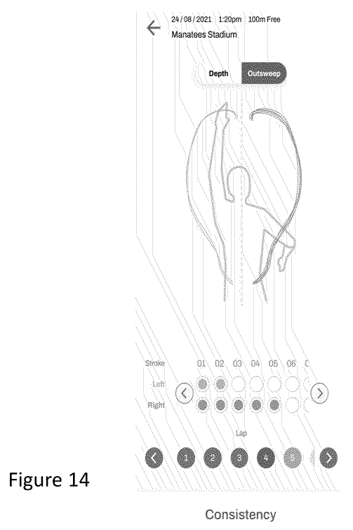

FIG. 8 shows depth and outsweep but a number of strokes for each hand are mapped to visually display consistency between the two hands. Notably, FIGS. 13 and 14 show example graphical representations of consistency between two hands in depth and outsweep views respectively.

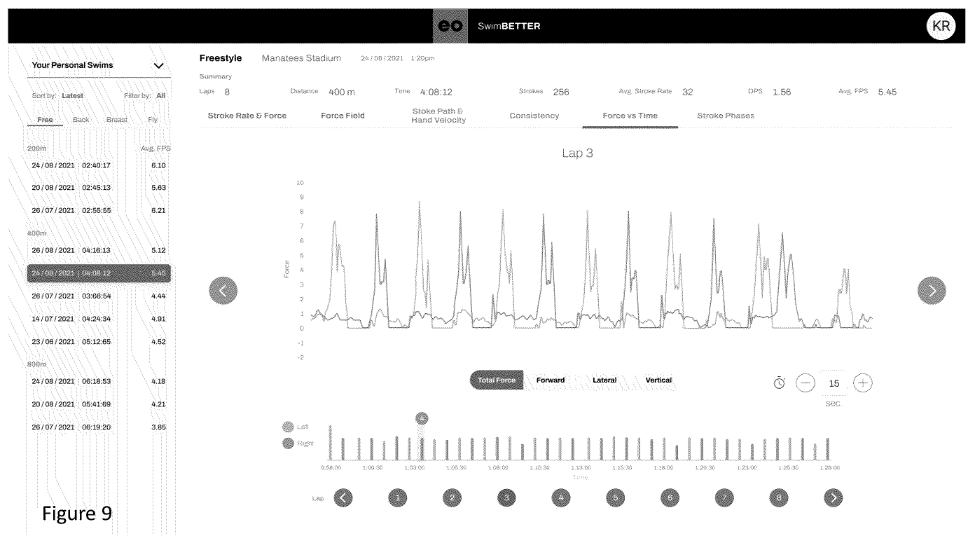

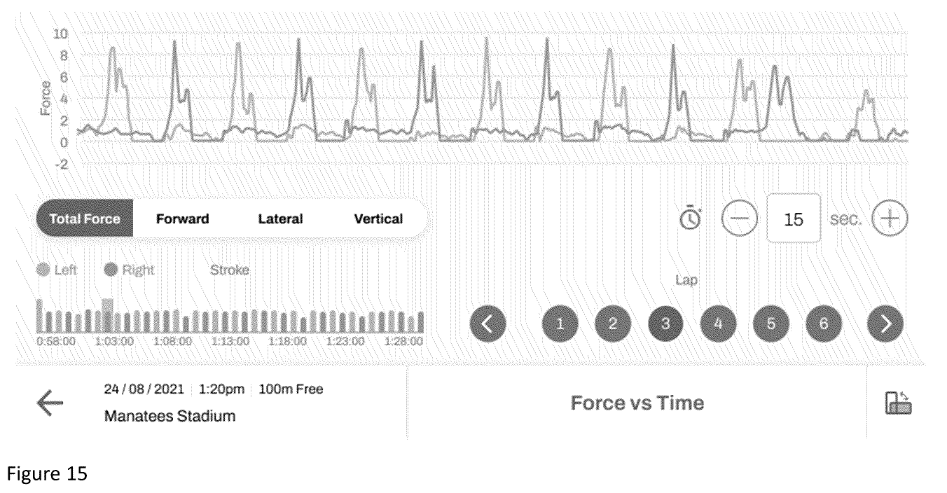

FIGS. 9 and 15 show examples of a graphical representation of force against time mapped for each hand. Notably, force can be segmented into total force or force in different directions such as forward, lateral and vertical directions.

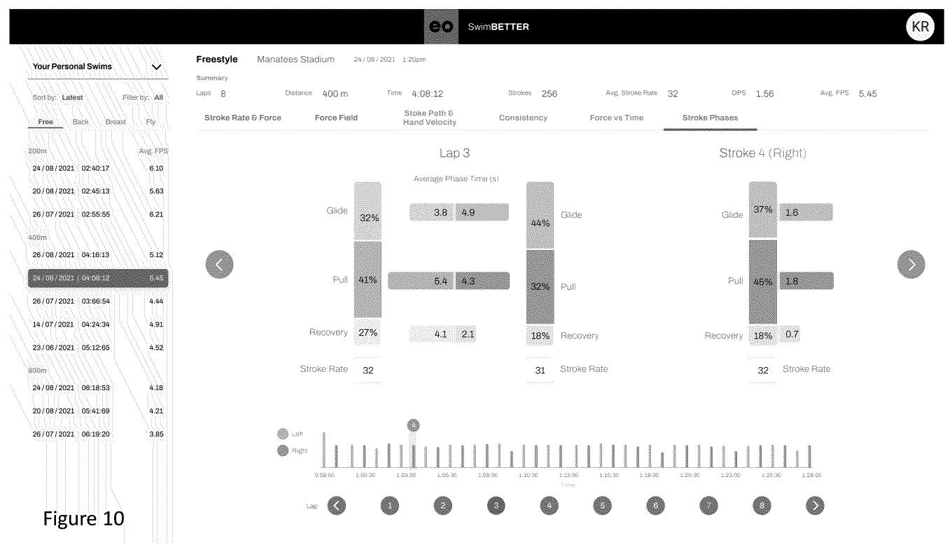

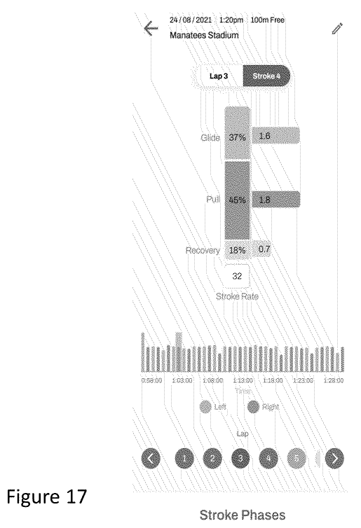

FIGS. 10, 16, and 17 are examples of another graphical representation where the stroke phases for each hand are broken down between what percentage of the stroke the hand is in the glide phase, pull phase and recovery phase. The segments can also include their respective time. FIG. 17 specifically shows the segmentation of stroke phases for one hand only.

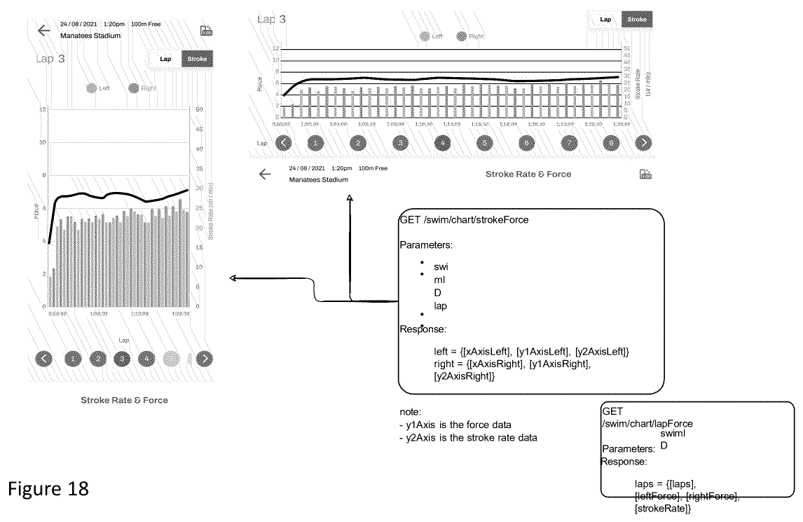

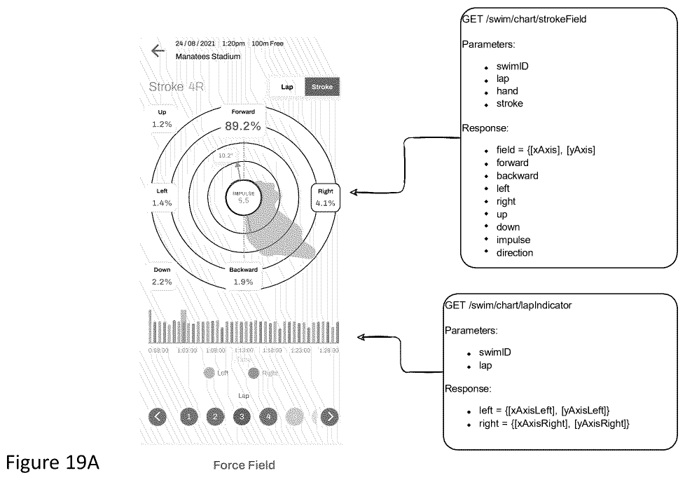

FIGS. 18 to 22C show example functions that can be applied in the system/method described herein to generate the graphical representations of various performance metrics.

FIG. 18 shows an example of the stroke rate and force being plotted on a graphical representation, where if a user has a unique identifier for the swimmer, such as a SwimID or the like, and can select the lap that is to be analysed, the system/method described herein can generate a graphical representation showing time on the x-axis and two y-axes, one showing force rate data (y1axis) and one showing stroke rate data (y2axis). By entering the SwimID, the user can also be shown a graphical representation of the laps, force in various directions and for each limb (such as left and right hand) as well as stroke rate.

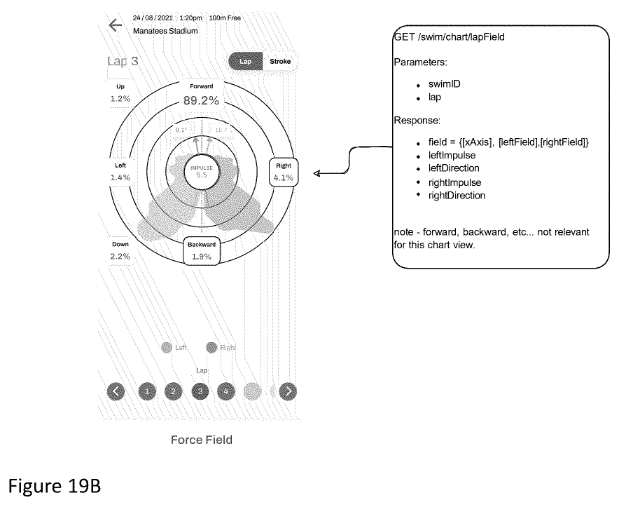

FIG. 19A shows an example of the functions that can be used to generate a graphical representation of the force field for one hand in a stoke. Typically, the user needs to provide/select the SwimID, lap, hand and stroke and the graphical representation generated can show the force generated over time as plotted on an x and y axis as well as the force generated in different directions including forward, backward, left, right, up, down, and the impulse. FIG. 19B shows an example of the force field generation for a lap, where when the SwimID and lap are selected by the user, the force field per hand can be generated showing the left and right impulse and direction respectively.

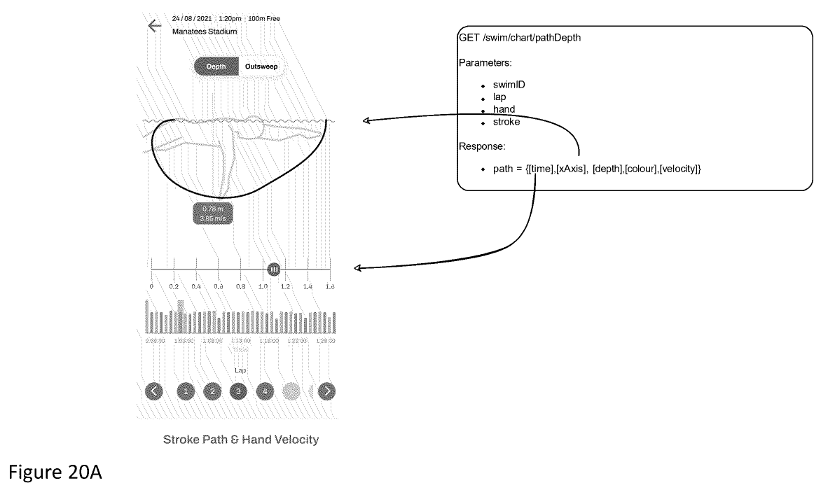

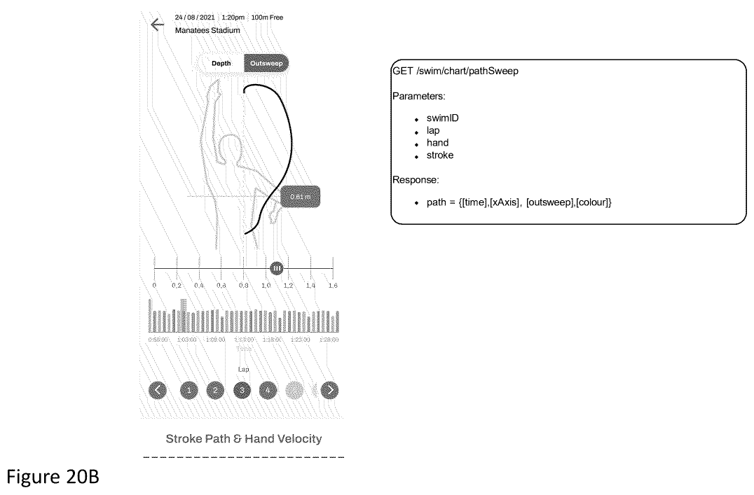

FIG. 20A shows an example graphical representation of the stroke path and hand velocity depth which can be generated through the user selection of the parameters SwimID, lap, hand, and stroke. The path generated is a function of:

$$path = \{ [ time ] , [ xAxis ] , [ depth ] , [ colour ] , [ velocity ] \} .$$

FIG. 20B shows an example graphical representation of stroke path and velocity showing outsweep generated by the selection of parameters including SwimID, lap, hand an stroke. The path generated in this example is typically:

$$path = \{ [ time ] , [ xAxis ] , [ outsweep ] , [ colour ] \} .$$

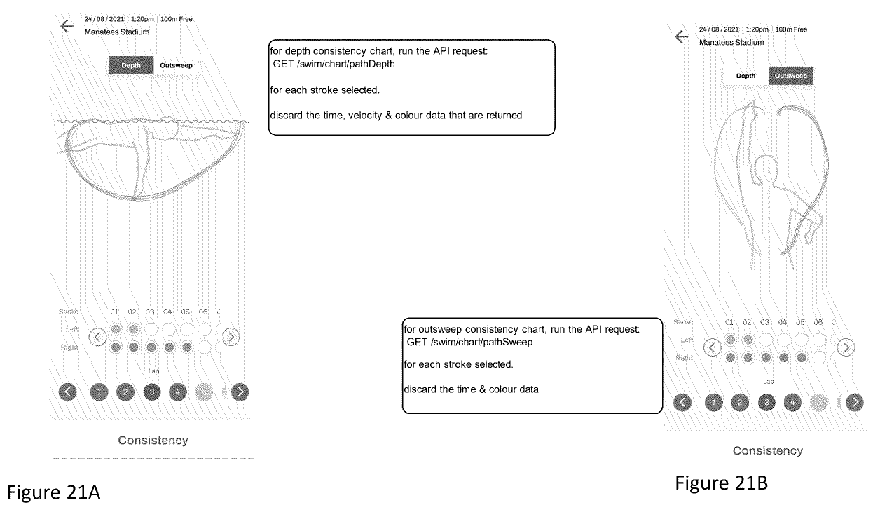

FIG. 21A shows an example graphical representation of depth consistency for a stroke, where typically time and velocity data are not required. Similarly FIG. 21B shows an example graphical representation of outsweep consistency for a stroke where time data is disregarded.

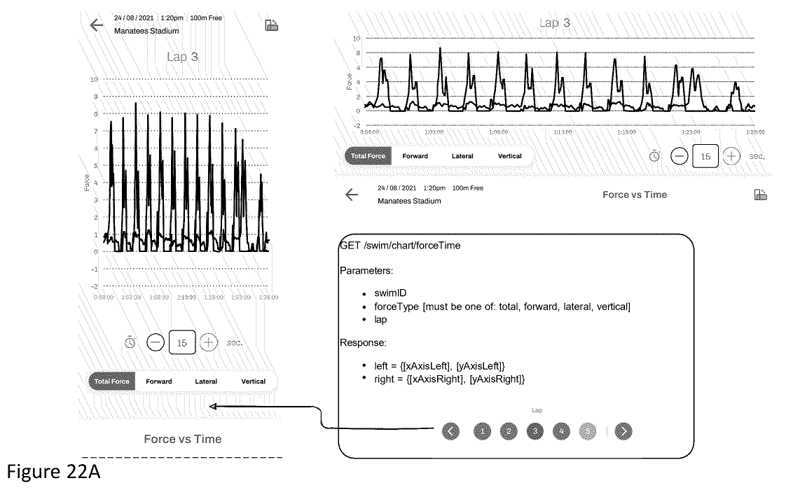

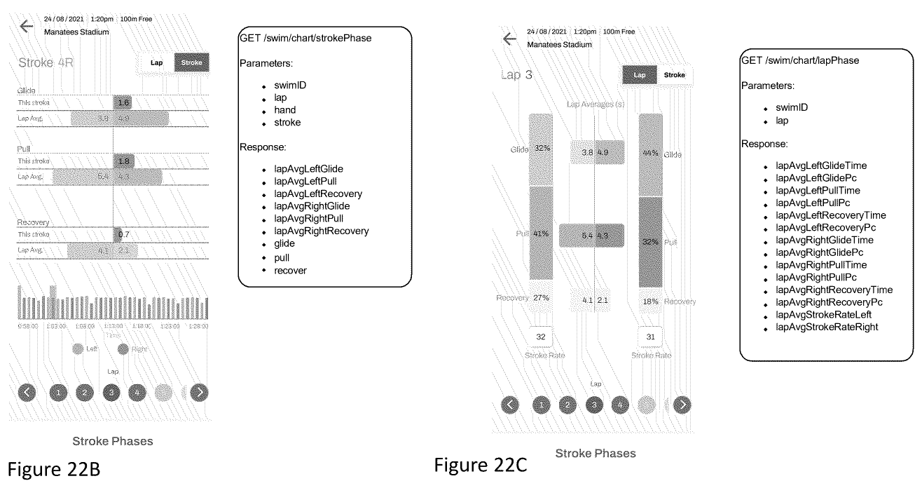

FIG. 22A shows an example graphical representation of time vs force for one or more limbs based on the SwimID and lap selected by the user, and further whether the user would like to see total force, or force in a particular direction (for example, forward, lateral or vertical)

FIG. 22B shows an example graphical representation of stroke phases segmented to determine what percentage or portion of a stroke is in a particular phase. Thus, for example, if a user provides/selects SwimID, lap, hand and stroke, they can see what percentage of the stroke of the left and right hand respectively was in glide, pull and recovery mode (average as well as pinpointed in time for a particular stroke).

Similarly FIG. 22C shows an example graphical representation of stroke phase segmentation in a lap. Thus, by selecting the SwimID and lap, the system/method can provide a representation of the respective left and right average glide time and percentage, average pull time and percentage, and average recovery time and percentage, and the stroke rate for the left and right limbs.

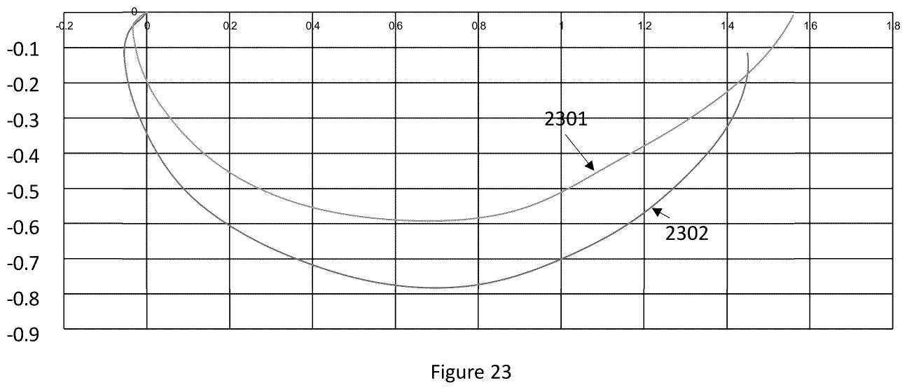

Further examples of graphical representations showing how the data generated can be analysed and compared are shown in FIGS. 23 to 33.

FIG. 23 shows an example of a graphical representation of a stroke comparison between a good stroke at 2301 and a worse stroke at 2302, where the swimmer swimming freestyle is pressing down during a catch phase of the stroke.

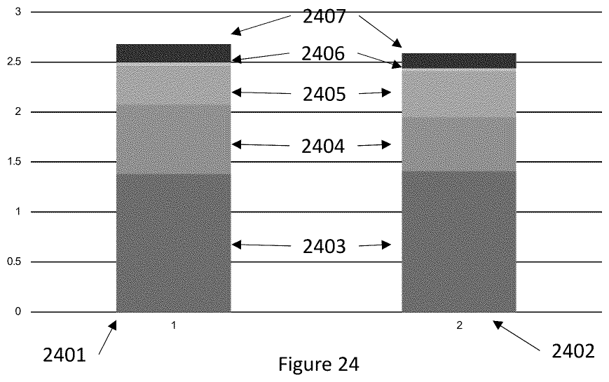

FIG. 24 shows an example graphical representation of a breakdown of the total force measured by a sensor during a stroke. In this example, the total force has been divided into six directions, where the better stroke is 2402 and the worse stroke is shown at 2401. In this breakdown of the stroke, at 2404 the band shows the amount of downward force detected in the each stroke, note the worse stroke has more downward force, whereas all other force directions the magnitude is the same across the two strokes. In particular, 2403 shows the forward propulsive force, 2405 shows the inward force, 2406 shows drag (palm pointing forward), and 2407 shows the outward force. Notably, in this particular stroke, what would be an upward force between 2406 and 2407 is almost negligible.

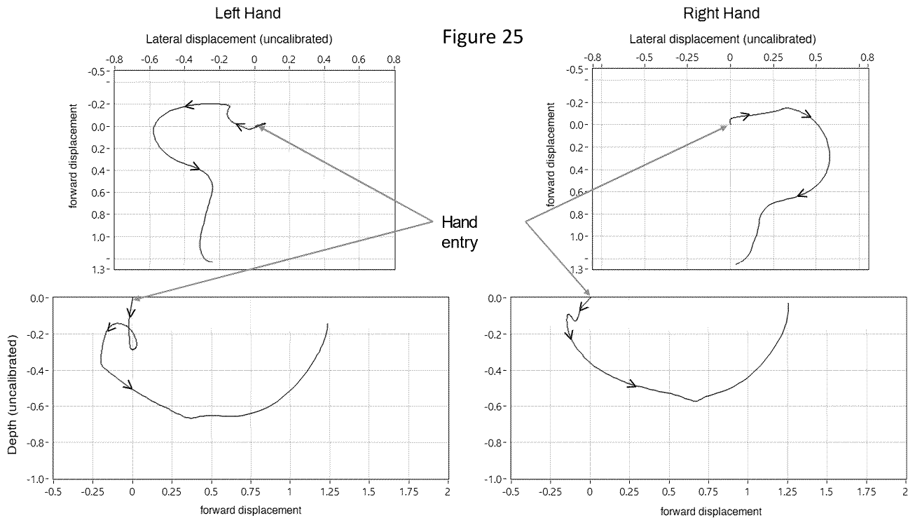

FIG. 25 shows examples of a graphical representation of the path of the Left and Right hand from top down view and side-on views each. As shown in the top-down view, the wide sweep in the right hand is evident, which can be confirmed in a videographic (or video) representation of the swim (as further discussed below).

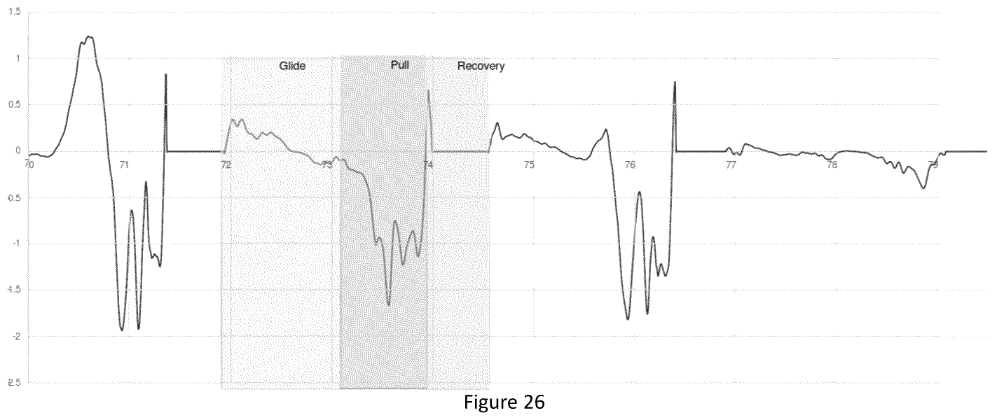

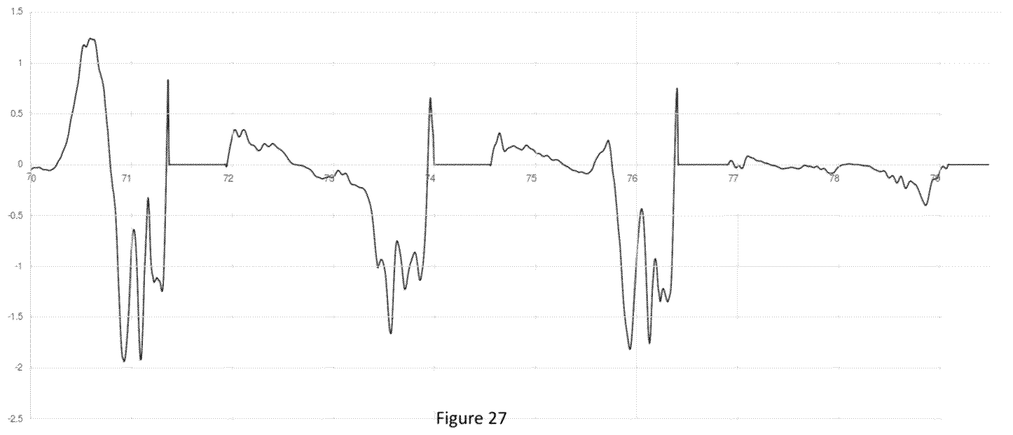

FIGS. 26 and 27 show a graphical representation of a trace indicating sideways pressure of a stroke. In this example, if the trace is below zero it shows outward pressure. Above zero is inward pressure. In FIG. 26, the shaded regions show the glide phase where time the hand lingers out front after entering the water, the pull phase where the time from when the swimmer begins to pull backward until the hand exits the water, and the recovery phase where the time the hand is out of the water. Note in FIG. 26, the pull phase begins when the hand starts the really wide outsweep.

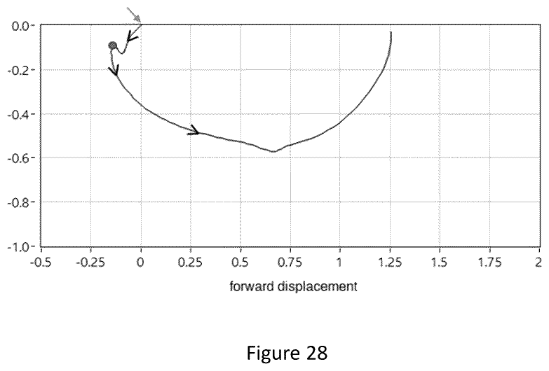

FIG. 28 is a graphical representation showing an example of a side on view of a swimmer's hand path showing the hand entering the water, proceeding down slightly and then coming up prior to the pull beginning.

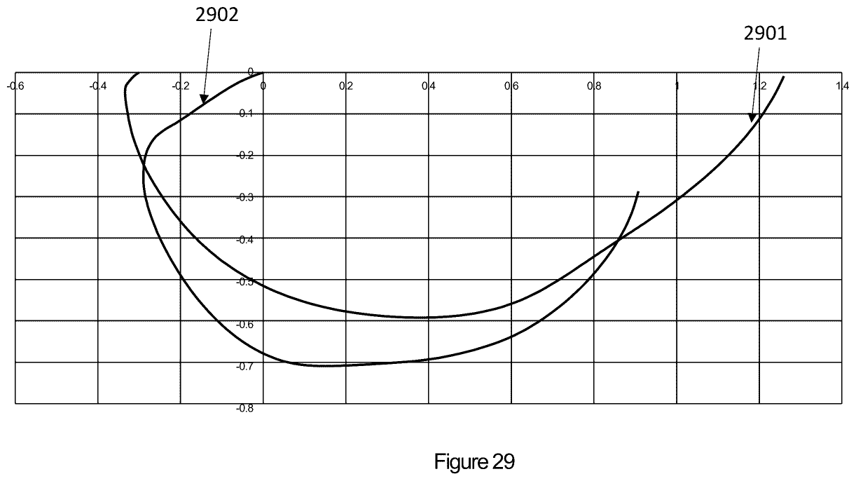

FIG. 29 is another example of a comparison of a good stroke 2901 and a bad stroke 2902. In this example, at 2902, the swimmer's hand enters too early (near her ears), then follows a downward & forward path. In contrast, 2901 shows the swimmer reaching further forward while the hand is out of the water. The hand spends less time pushing through the water before the pull phase begins.

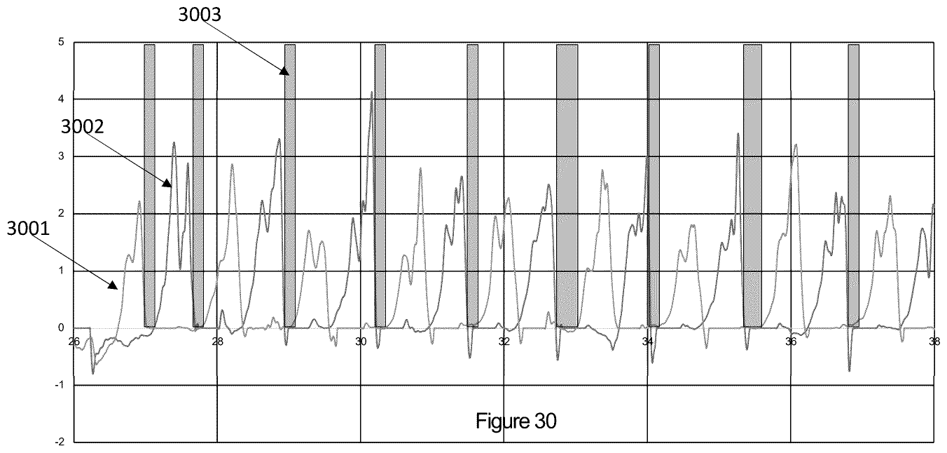

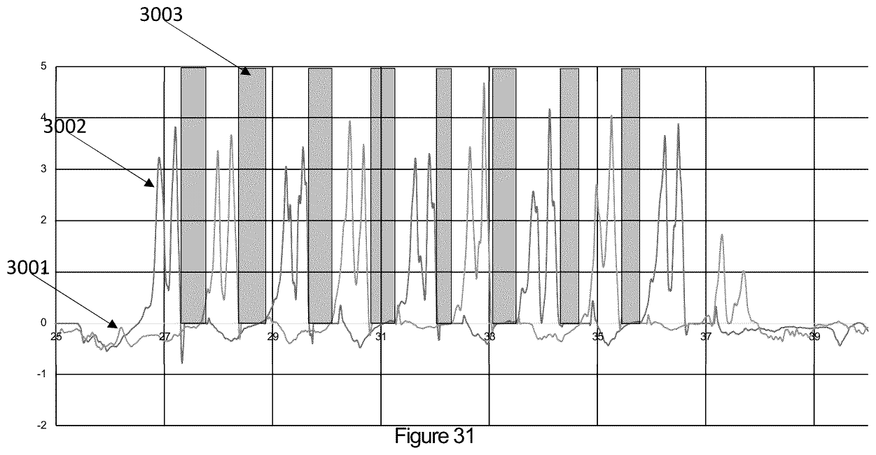

FIGS. 30 and 31 show an example of the propulsive forces for the right hand at 3001 and left hand 3002 for a swimmer. The regions 3003 are dead bands, a period of time where the swimmer is not generating any forward propulsion. The swimmer is effectively dead in the water at these points. Note that the swimmer in FIG. 31 has much larger dead bands than the swimmer in FIG. 30. Thus, the present system and method can allow for a comparison of swimmers for any of the performance metrics described herein.

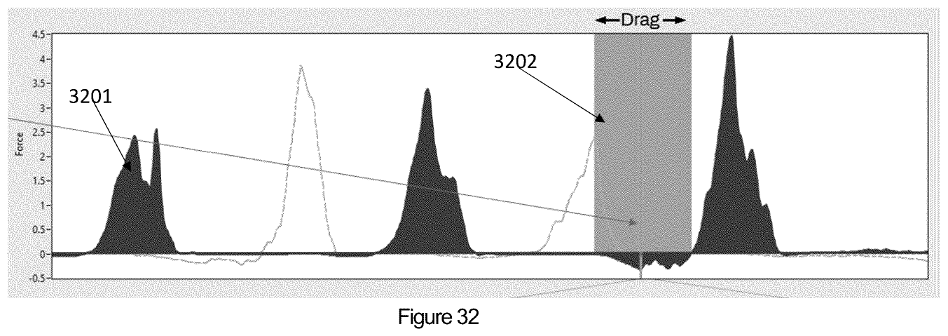

In the graphical representation example of FIG. 32, the trace 3201 shows the amount of forward propulsion detected for a swimmer and the shaded region 3202 is the glide phase, where the hand enters the water before the pull begins. Notably, when the trace 3201 goes negative it's an indication the palm of the hand is pointing forward causing a lot of drag. Notably, it is apparent from this representation that for this particular swimmer there is much less drag in a previous stroke.

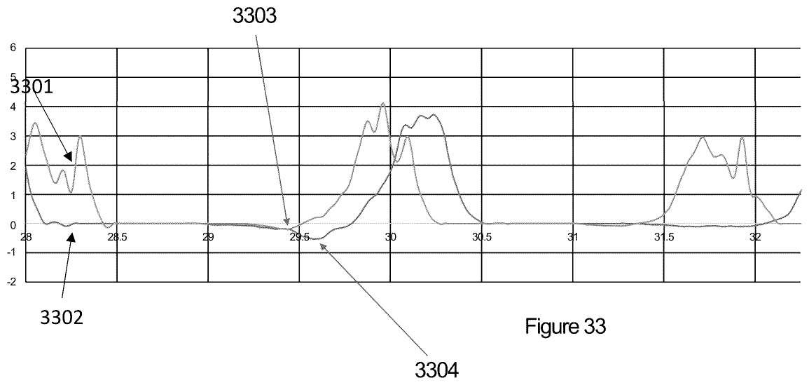

Referring now to FIG. 33, this is an example of a graphical representation of a swimmer where the trace 3302 shows forward propulsion of a bad stroke, whereas trace 3301 is same swimmer swimming normal. In this example, both strokes are “breathing” strokes where the swimmer takes a breath. In the good stroke, the forward propulsion starts faster, the swimmer takes their breath quickly as shown at 3303. In contrast, the late timing of the breath in the bad stroke at 3304, delays when the swimmer generates forward propulsion.

Further examples of how the metrics displayed in FIGS. 5 to 36 can be used by coaches/trainers or the swimmers themselves to improve their swimming techniques is provided below:

### Example: Overextension of Hand and Resultant Body Roll-Shown for Example in FIG. 23

The length at the front of a stroke is vital but too much reach actually doesn't add significantly to the real length of the swimmer's stroke and it can cost stroke rate, loss of rhythm and disengage the connection between the swimmer's hand and body. An overextension of the arm and the excessive body roll with the hand reaching out too far and facing slightly upward at the front can cause the athlete to disconnect from their stroke and interrupt their rhythm. The hip will typically roll too far and it is common to see swimmers over rotating in order to achieve more length. This puts the swimmer off balance. An unbalanced athlete will compromise their ability to produce force in any sport. This excess roll compromises power and depth at the back of the stroke while delaying the propulsive phase at the front. The lack of balance shows up in several ways-typically the legs crossover and attempt to rebalance the body. Another consequence of the roll and indication of the lack of balance is the upward facing hand at the front of the stroke. It is so important to have the correct amount of body roll that enables the swimmer to anchor early in their catch phase.

Referring more specifically to FIGS. 7, 8, 11, 12 13, 14 20, and 21, on the stroke path chart in the hand depth sample shows the side profile of the hand as it moves through the water. At the beginning of the stroke, the hand enters and then go up towards the surface before beginning the catch.

Thus, in one graphical representation that can be generated, force can be filtered into force generated in six directions, and forward propulsion can be graphed according. As an example, FIG. 9 in particular displays the following 4 view types: total force measured left/right hands; forward (positive) propulsive forces, and backward drag forces which would be negative in value; Forces applied downward (negative), or upward (positive); and, Forces applied inward (positive), or outward (negative) directions.

Furthermore, the graph can be generated which show where the hand enters the glide phase and propulsive force goes negative for a short period reflecting the upward movement of the hand; in other words, generating drag.

### Example: Stroke Rate and Timing

The relationship between stroke rate and speed is critical in freestyle. An athlete may be “efficient” in terms of length and travel but there typically needs to be the right balance between stroke rate and stroke length. Getting caught in a situation where the athlete's hand waits at the front of their stroke for too long whilst the other hand recovers is a common problem creating a ‘dead spot’ in a swimmer's stroke. Minimising this ‘dead spot’ is critical to find the best timing for each athlete. Typically, the right arm extends into the glide phase and waits for the left arm to enter before it commences the propulsive phase.

Referring more specifically to FIGS. 9, 15, and 22A, to help identify this problem, using the system and method described herein, coaches are able to measure the gap between the propulsive phases of each arm. Each athlete has a timing that will work best for them. Typically, too much gap costs momentum, rhythm and speed. Further, the time of the glide phase from entry to catch and how this relates to stroke rate can also be measured as stroke rate improvements can lead to speed gains.

The method and system described herein can provide an indication of power per stroke and can monitor how a swimmer's power increases as their timing improves.

### Example: Breathing and Hip Timing-Shown for Example in FIG. 33:

Typically, a fast time means a fast swim. In order to swim faster the swimmer typically has to be balanced, because propulsion comes with balance and a stable body will produce more force. If a swimmer breathes through the propulsive phase, their body in freestyle is typically on its side and their trunk struggles to align and to connect through its core. The key is to get the swimmer to breathe out of the way. That is, minimising their breath can reduce their body roll and allow them to reach a balanced position earlier and for longer.

The system and method described herein can show or generate an imaging which shows a lower force at the front of the stroke. As a consequence every breathing stroke has less travel and a shorter impulse—stroke after stroke there is a compromise in length. A cost in length is a cost in speed. Thus, if this is corrected, the swim time can become faster.

Example: Pressing Down During Catch (Poor Catch)—Shown for Example in FIGS. 23 and 24, where the Swimmer is Pressing Down

The catch is the key to the power of good freestyle swimming as getting the catch right can set up a swimmer's stroke in the underwater phase. It is important to master the feel of the water in a relaxed manner, so after entering and reaching forwards, a swimmer's hand and forearm are pressing primarily backwards as they move towards their hip to exit the water. A common fault is failing to get a correct catch where a swimmer is pressing primarily downwards with the hand towards the bottom of the pool, rather than backwards.

Visually the data can thus show a spike in down force on the right hand during the catch phase, which makes up too large a portion of the total force for each stroke. This can compromise forward propulsive force and minimises performance.

### Example: Large Outsweep—Shown for Example in FIGS. 25-27:

Swimming freestyle effectively comes from not only holding the right body position but also ensuring that a swimmer's arms are moving in the right path. One of the common faults in many swimmers is a large outward sweep (or outsweep) action in the catch phase When the swimmer sweeps too far outside the body, this can limit any effective propulsion in the catch position—with a sideways force instead of a propulsive force. It is important to get the correct catch position to enable the swimmer's smooth trajectory forward in a straight line and not putting the arm in a weaker position with the hand wider than the elbow. The excessive width of stroke compromises how far a swimmer can travel with each stroke which can ultimately affect their speed.

Outsweep is shown as an example in FIGS. 7A, 7B, 8, 12, 14, 20 and 21. Mapping the hand and arm movement in this way can assist in identifying a large outsweep, where the top of the chart is where the hand first starts the catch and the bottom is where the hand exits the water. The data can also show a spike in the j lateral force (outwards) during the catch.

### Example: No Reach Phase-Shown for Example in FIGS. 29-30, Showing No Reach:

Having the right amount of reach in a stroke is important for propulsion. Typically, there will be a marked difference with those who enter the water with a deep downwards action, and those who are reaching forwards at shoulder depth which ensures their hand and forearm is setup to begin the underwater part of the stroke—the catch. A deep downwards action during the reach phase of the stroke can create a lot more drag, during what is normally the fastest and most efficient point of the stroke, thus, missing out on the initial setup phase of the all important catch.

Visually, the data can show the hand going deep through the front of the stroke compared to a swimmer who reaches correctly at shoulder depth. The data also shows a larger gap between the power phases of the left and right hand which may result in a faster stroke rate however this is at the cost of effective power generated, which means the swimmer gets a lot less out of each stroke. It will be appreciated that once the reach and catch are mastered, then the freestyle swim can become that much smoother, stronger and more importantly, much faster.

### Example: Angle of Attack

The system/method described herein can determine the angle a limb such as a swimmer's hand is facing at a particular point in time, and the pressure that was being exerted at that time or moment in a stroke. Thus, the system/method can determine how much of the hand was pointing at the feet, or sideways and further, how much force was being generated at that particular angle.

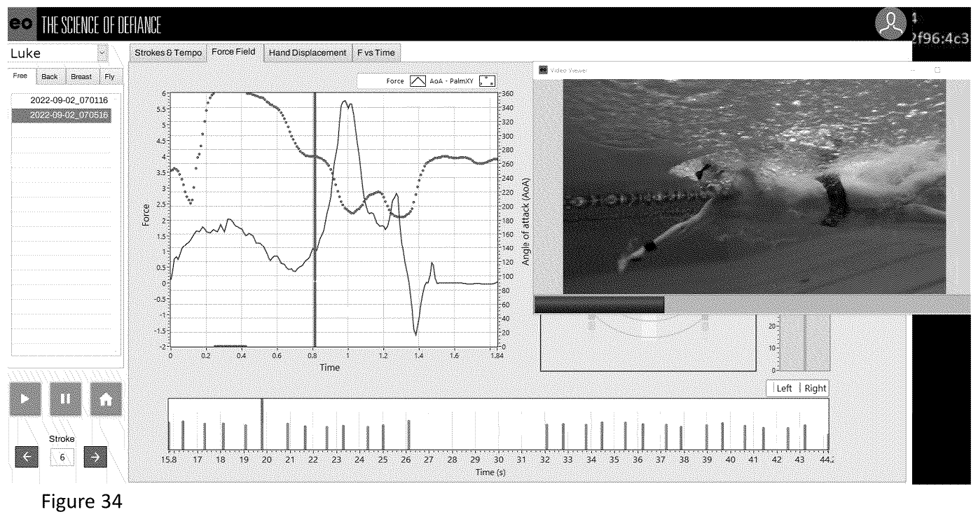

FIG. 34 shows an example of the angle of attack being graphed and further a video of the swimmer at that particular point in time being linked to and superimposed on the graph that has been generated. Accordingly, it will be appreciated that a coach or any user can see how much force has been generated by a swimmer at a particular angle of attack and they may be able to determined improvements that can be made by the swimmer during their swim. For example, they may be able to determine that by the middle of the swim, the swimmer is starting to fatigue and can see that the angle at which their hand is performing the stroke is not ideal for the amount of force that is being applied. The visualisation of the angle of attack in a graphical interface also means that a coach or user does not have to watch the entirety of the swimmer's video but can very quickly determined where the issues in technique have developed and skip to that part or time interval in the video.

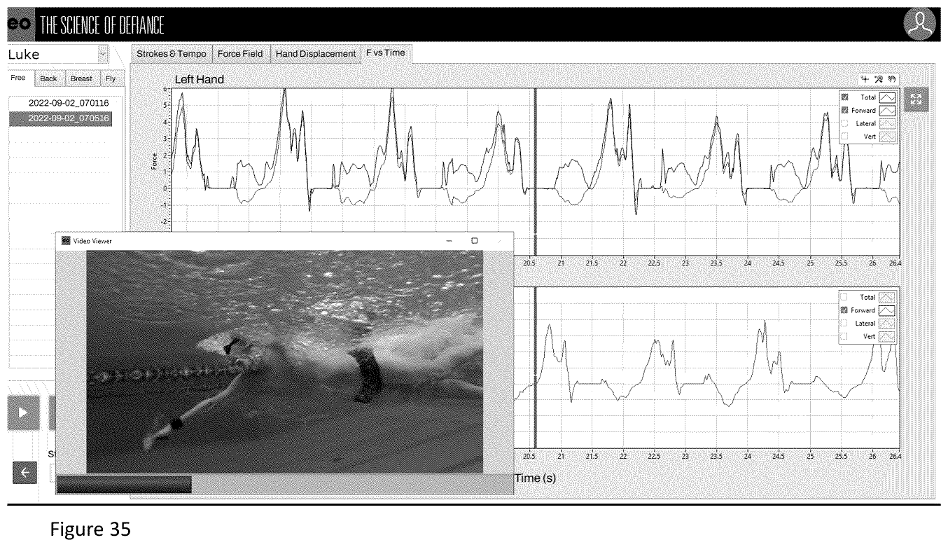

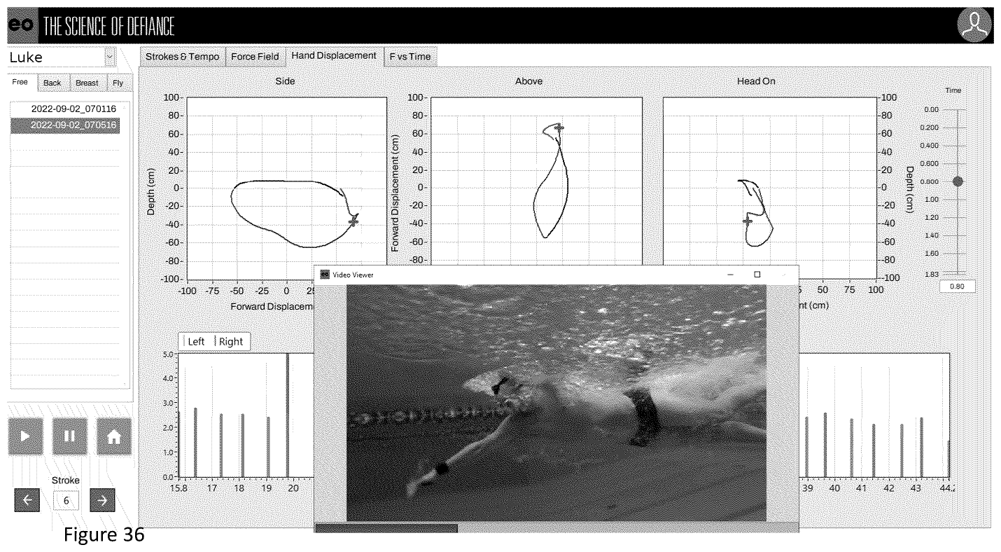

Notably, video image can be superimposed and linked to any performance metric described herein. FIG. 35 shows an example of video linked to graphs showing force vs time of the left hand of a swimmer. FIG. 36 shows an example of a video linked to hand displacement of a swimmer, where the graphical interface shows hand displacement from a side view, from above and from the head on view of the swimmer.

Thus, the system/method described herein can allow athletes to further improve their techniques by considering various performance metrics which can be displayed graphically to a user and mapped to a user's swim record for ease of reference.

The term comprise” and variants of that term such as “comprises” or “comprising” are used herein to denote the inclusion of a stated integer or integers but not to exclude any other integer or integers, unless in the context or usage an exclusive interpretation of the term is required.

Those skilled in the art will appreciate that the invention described herein is susceptible to variations and modifications other than those specifically described. All such variations and modifications are to be considered within the scope and spirit of the present invention the nature of which is to be determined from the foregoing description.

1. A method of determining a performance metric of an athlete, the method including the steps of, in a processing system:
receiving data from at least one limb of the athlete;
determining an orientation of the at least one limb and applying the orientation to the received data; and
generating the performance metric of the at least one limb based on received data and orientation of the at least one limb.

2. The method of claim 1, the method includes receiving data including at least one of:
pressure data from the at least one limb;
acceleration data of the at least one limb; and
time data.

3. The method of claim 2, wherein the at least one limb is a hand of the athlete; and wherein the pressure data includes receiving palm pressure of the hand and side pressure of the hand.

4. The method of claim 3, wherein the method includes determining a pressure difference; and wherein the pressure difference being difference in pressure between the palm pressure and the side pressure.

5. The method of claim 3, wherein receiving pressure data includes receiving data from a left hand and a right hand of the athlete.

6. The method of claim 1, wherein determining the orientation of the athlete includes:
receiving location data; and
applying a rotation function to at least some of the received data to orientate the athlete in accordance with the location.

7. The method of claim 6, wherein applying the rotation function includes applying a quaternion rotation.

8. The method of claim 2, wherein the method includes determining at least one of:
pressure in three-dimensions; and
acceleration in three-dimensions.

9. The method of claim 8, wherein determining pressure in three dimensions includes determining:
forward pressure of the limb;
lateral pressure of the limb; and
vertical pressure of the limb.

10. The method of claim 2, wherein determining acceleration in three dimensions includes determining:
forward acceleration of the limb;
lateral acceleration of the limb; and
vertical acceleration of the limb.

11. The method of any one of claim 2, wherein the method includes determining velocity of the limb in at least one dimension including at least one of forward velocity, lateral velocity, and vertical velocity.

12. The method of claim 11, wherein the method of determining velocity includes:
determining acceleration in at least one dimension; and
integrating the acceleration in at least one dimension to determine velocity in at least one dimension.

13. The method of claim 11, wherein the method includes determining displacement of the limb in at least one dimension, including at least one of forward displacement, lateral displacement, and vertical displacement.

14. The method of claim 13, wherein determining displacement in at least one dimension includes integrating the velocity in at least one dimension.

15. The method of claim 1, wherein the athlete is a swimmer.

16. The method of claim 15, wherein the method includes detecting a stroke event by identifying an entry point of a hand and an exit point of the hand.

17. The method of claim 16, wherein identifying the entry point and the exit point includes determining a pressure difference between pressure measured on a side of the hand and pressure measured by a palm of the hand, and a time period.

18. The method of claim 15, wherein the method includes detecting a lap event by identifying a change in forward direction.

19. The method of claim 18, wherein identifying a change in forward direction includes determining forward pressure and a time period.

20. The method of claim 15, wherein the method includes detecting a pull event.

21. The method of claim 20, wherein detecting pull includes identifying at least one position within a stroke where a forward velocity is at least one of zero and about zero, indicating a transition from catch to pull.

22. The method of claim 16, wherein the method includes aggregating the stroke event, the lap event, and the pull event for a time period.

23. The method of claim 22, wherein the method includes generating a graphical representation of at least one of the stroke event, lap event and pull event for at least one time period for the swimmer.

24. The method of claim 15, wherein the method includes determining stroke type or swim style.

25. The method of claim 24, wherein stroke type or swim style can include at least one of freestyle, backstroke, breaststroke, butterfly, and drills.

26. The method of claim 1, wherein the method includes generating a graphical representation of at least one performance metric.

27. The method of claim 26, wherein the athlete is a swimmer; and wherein method includes generating at least one graphical representation including at least one:
stroke rate and force over time;
force over time showing force applied by at least one limb of the athlete at a particular time period;
stroke path of the at least one a limb over a time period;
velocity of the at least one limb over a time period;
stroke path of at least two limbs for comparison over a time period;
segmentation of stroke phases at a time period; and
angle of attack.

28. The method of claim 27, wherein stroke rate includes strokes per minute over time.

29. The method of claim 27, wherein the graphical representation of force over time includes at least one of force per, stroke force field for a limb, and force versus time.

30. The method of claim 27, wherein the stroke path includes depth and outsweep of the at least one limb.

31. The method of claim 27, wherein the comparison includes determining consistency between limbs in relation to at least one of movement through the water, depth, and outsweep.

32. The method of claim 27, wherein segmentation of stroke phases includes generating a graphical representation showing the percentage of glide, pull, and recovery phases of a stroke.

33. The method of claim 27, wherein the angle of attack includes determining the angle of a limb at a particular point in time, and the pressure that was being exerted at that time.

34. A system for determining a performance metric of an athlete, the system including a sensing device, and a processing system being configured to:
receive data from the sensing device being attached to at least one limb of the athlete;
determine an orientation of the at least one limb and applying the orientation to the received data; and
generate the performance metric of the at least one limb based on received data and orientation of the at least one limb.

35. A processing system for determining a performance metric of an athlete, the processing system being configured to:
receive data from the sensing device being attached to at least one limb of the athlete;
determine an orientation of the at least one limb and applying the orientation to the received data; and
generate the performance metric of the at least one limb based on received data and orientation of the at least one limb.
